SEQUENCE LISTING (91 sequences)

SEQ ID NO: 1 - DNA, 321 bp, Artificial Sequence
  Synthetic construct
gatatccagatgacccagtccccgagctccctgtccgcctctgtgggcgatagggtcaccatcacctgccgtgccagtcaggatgtgaataccgctgtagcctggtatcaacagaaaccaggaaaagctccgaagcttctgatttactcggcatccttcctctactctggagtcccttctcgcttctctggtagccgttccgggacggatttcactctgaccatcagcagtctgcagccggaagacttcgcaacttattactgtcagcaacactacaccaccccaccaacgttcggacagggtaccaaggtggagatcaaa

SEQ ID NO: 2 - protein, 107 aa, Artificial Sequence [sequence omitted]

SEQ ID NO: 3 - DNA, 360 bp, Artificial Sequence
  Synthetic construct
gaggttcagctggtggagtctggcggtggcctggtgcagccagggggctcactccgtttgtcctgtgcagcttctggcttcaacattaaggacacctacattcactgggtgcgtcaggccccgggtaagggcctggaatgggttgcacgcatctacccaaccaatggctacacccgctatgccgatagcgtcaagggccgtttcactataagcgcagacacatccaaaaacacagcctacctacaaatgaacagcttaagagctgaggacactgccgtctattattgtagccgctggggcggcgacggcttttacgccatggactactggggtcaaggaaccctggtcaccgtctcctcg

SEQ ID NO: 4 - protein, 120 aa, Artificial Sequence [sequence omitted]

SEQ ID NO: 5 - DNA, 45 bp, Artificial Sequence
  Synthetic construct
  misc_feature (18)..(18): n is a, c, g, or t
  misc_feature (24)..(24): n is a, c, g, or t
cctgccgtgccagtcagnhtgtgnhtaccgctgtagcctggtatc

SEQ ID NO: 6 - DNA, 45 bp, Artificial Sequence
  Synthetic construct
  misc_feature (18)..(18): n is a, c, g, or t
  misc_feature (27)..(27): n is a, c, g, or t
cgaagcttctgatttacnhtgcatccnhtctctactctggagtcc

SEQ ID NO: 7 - DNA, 45 bp, Artificial Sequence
  Synthetic construct
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
cttattactgtcagcaanhtnhtnhtnhtccaccaacgttcggac

SEQ ID NO: 8 - DNA, 48 bp, Artificial Sequence
  Synthetic construct
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
  misc_feature (30)..(30): n is a, c, g, or t
cttattactgtcagcaanhtnhtnhtnhtnhtccaccaacgttcggac

SEQ ID NO: 9 - DNA, 51 bp, Artificial Sequence
  Synthetic construct
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
  misc_feature (30)..(30): n is a, c, g, or t
  misc_feature (33)..(33): n is a, c, g, or t
cttattactgtcagcaanhtnhtnhtnhtnhtnhtccaccaacgttcggac

SEQ ID NO: 10 - DNA, 54 bp, Artificial Sequence
  Synthetic construct
  misc_feature (18)..(18): n is a, c, g, or t
  misc_feature (21)..(21): n is a, c, g, or t
  misc_feature (24)..(24): n is a, c, g, or t
  misc_feature (27)..(27): n is a, c, g, or t
  misc_feature (30)..(30): n is a, c, g, or t
  misc_feature (33)..(33): n is a, c, g, or t
  misc_feature (36)..(36): n is a, c, g, or t
cttattactgtcagcaanhtnhtnhtnhtnhtnhtnhtccaccaacgttcggac

SEQ ID NO: 11 - DNA, 40 bp, Artificial Sequence
  Synthetic construct
  misc_feature (16)..(16): n is a, c, g, or t
  misc_feature (22)..(22): n is a, c, g, or t
ggcttcaacattaagnhtaccnhtattcactgggtgcgtc

SEQ ID NO: 12 - DNA, 56 bp, Artificial Sequence
  Synthetic construct
  misc_feature (16)..(16): n is a, c, g, or t
  misc_feature (22)..(22): n is a, c, g, or t
  misc_feature (28)..(28): n is a, c, g, or t
  misc_feature (31)..(31): n is a, c, g, or t
  misc_feature (37)..(37): n is a, c, g, or t
ctggaatgggttgcanhtatcnhtccanhtnhtggcnhtacccgctatgccgatag

SEQ ID NO: 13 - DNA, 46 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
ctattattgtagccgctggnhtnhtnhtnhtgccatggactactgg

SEQ ID NO: 14 - DNA, 49 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
ctattattgtagccgctggnhtnhtnhtnhtnhtgccatggactactgg

SEQ ID NO: 15 - DNA, 52 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
ctattattgtagccgctggnhtnhtnhtnhtnhtnhtgccatggactactgg

SEQ ID NO: 16 - DNA, 55 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
  misc_feature (38)..(38): n is a, c, g, or t
ctattattgtagccgctggnhtnhtnhtnhtnhtnhtnhtgccatggactactgg

SEQ ID NO: 17 - DNA, 58 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
  misc_feature (38)..(38): n is a, c, g, or t
  misc_feature (41)..(41): n is a, c, g, or t
ctattattgtagccgctggnhtnhtnhtnhtnhtnhtnhtnhtgccatggactactgg

SEQ ID NO: 18 - DNA, 61 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
  misc_feature (38)..(38): n is a, c, g, or t
  misc_feature (41)..(41): n is a, c, g, or t
  misc_feature (44)..(44): n is a, c, g, or t
ctattattgtagccgctggnhtnhtnhtnhtnhtnhtnhtnhtnhtgccatggactactgg

SEQ ID NO: 19 - DNA, 64 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
  misc_feature (38)..(38): n is a, c, g, or t
  misc_feature (41)..(41): n is a, c, g, or t
  misc_feature (44)..(44): n is a, c, g, or t
  misc_feature (47)..(47): n is a, c, g, or t
ctattattgtagccgctggnhtnhtnhtnhtnhtnhtnhtnhtnhtnhtgccatggactactgg

SEQ ID NO: 20 - DNA, 67 bp, Artificial Sequence
  Synthetic construct
  misc_feature (20)..(20): n is a, c, g, or t
  misc_feature (23)..(23): n is a, c, g, or t
  misc_feature (26)..(26): n is a, c, g, or t
  misc_feature (29)..(29): n is a, c, g, or t
  misc_feature (32)..(32): n is a, c, g, or t
  misc_feature (35)..(35): n is a, c, g, or t
  misc_feature (38)..(38): n is a, c, g, or t
  misc_feature (41)..(41): n is a, c, g, or t
  misc_feature (44)..(44): n is a, c, g, or t
  misc_feature (47)..(47): n is a, c, g, or t
  misc_feature (50)..(50): n is a, c, g, or t
ctattattgtagccgctggnhtnhtnhtnhtnhtnhtnhtnhtnhtnhtnhtgccatggactactgg

SEQ ID NO: 21 - DNA, 27 bp, Artificial Sequence
  Synthetic construct
gmcatccagatgacccagtctccatcc

SEQ ID NO: 22 - DNA, 27 bp, Artificial Sequence
  Synthetic construct
gmcatccagttgacccagtctccatcc

SEQ ID NO: 23 - DNA, 27 bp, Artificial Sequence
  Synthetic construct
gccatccggatgacccagtctccatyc

SEQ ID NO: 24 - DNA, 29 bp, Artificial Sequence
  Synthetic construct
gacatccagatgacccagtctccatcttc

SEQ ID NO: 25 - DNA, 27 bp, Artificial Sequence
  Synthetic construct
gacatccagatgacccagtctccwtcc

SEQ ID NO: 26 - DNA, 30 bp, Artificial Sequence
  Synthetic construct
gaaattgtgttgacgcagtctccagscacc

SEQ ID NO: 27 - DNA, 30 bp, Artificial Sequence
  Synthetic construct
gaaatagtgatgacgcagtctccagccacc

SEQ ID NO: 28 - DNA, 27 bp, Artificial Sequence
  Synthetic construct
gaaattgtgttgacacagtctccagcc

SEQ ID NO: 29 - DNA, 24 bp, Artificial Sequence
  Synthetic construct
acactctcccctgttgaagctctt

SEQ ID NO: 30 - DNA, 39 bp, Artificial Sequence
  Synthetic construct
gaggtgcagctggtggagtctgggggaggcttggtccag

SEQ ID NO: 31 - DNA, 39 bp, Artificial Sequence
  Synthetic construct
caggtgcagctggtggagtctgggggaggcttggtcaag

SEQ ID NO: 32 - DNA, 39 bp, Artificial Sequence
  Synthetic construct
gaggtgcagctggtggagtctgggggaggcttggtaaag

SEQ ID NO: 33 - DNA, 39 bp, Artificial Sequence
  Synthetic construct
gaggtgcagctggtggagtctgggggaggtgtggtacgg

SEQ ID NO: 34 - DNA, 39 bp, Artificial Sequence
  Synthetic construct
caggtgcagctggtggagtctgggggaggcgtggtccag

SEQ ID NO: 35 - DNA, 39 bp, Artificial Sequence
  Synthetic construct
gaggtgcagctggtggagactggaggaggcttgatccag

SEQ ID NO: 36 - DNA, 22 bp, Artificial Sequence
  Synthetic construct
tgaggagacrgtgaccagggtg

SEQ ID NO: 37 - DNA, 21 bp, Artificial Sequence
  Synthetic construct
tgaagagacggtgaccattgt

SEQ ID NO: 38 - DNA, 22 bp, Artificial Sequence
  Synthetic construct
tgaggagacggtgaccagggtt

SEQ ID NO: 39 - DNA, 23 bp, Artificial Sequence
  Synthetic construct
tgaggagacggtgaccgtggtcc

SEQ ID NO: 40 - DNA, 40 bp, Artificial Sequence
  Synthetic construct
aaatgcctatgcatcggmcatccagatgacccagtctcca

SEQ ID NO: 41 - DNA, 40 bp, Artificial Sequence
  Synthetic construct
aaatgcctatgcatcggmcatccagttgacccagtctcca

SEQ ID NO: 42 - DNA, 40 bp, Artificial Sequence
  Synthetic construct
aaatgcctatgcatcggccatccggatgacccagtctcca

SEQ ID NO: 43 - DNA, 40 bp, Artificial Sequence
  Synthetic construct
aaatgcctatgcatcggacatccagatgacccagtctcca

SEQ ID NO: 44 - DNA, 40 bp, Artificial Sequence
  Synthetic construct
aaatgcctatgcatcggacatccagatgacccagtctccw

SEQ ID NO: 45 - DNA, 40 bp, Artificial Sequence
  Synthetic construct
aaatgcctatgcatcggaaattgtgttgacgcagtctcca

SEQ ID NO: 46 - DNA, 40 bp, Artificial Sequence
  Synthetic construct
aaatgcctatgcatcggaaatagtgatgacgcagtctcca

SEQ ID NO: 47 - DNA, 40 bp, Artificial Sequence
  Synthetic construct
aaatgcctatgcatcggaaattgtgttgacacagtctcca

SEQ ID NO: 48 - DNA, 53 bp, Artificial Sequence
  Synthetic construct
gaagatgaagacagatggtgcagccaccgtacgtttgatttccaccttggtcc

SEQ ID NO: 49 - DNA, 53 bp, Artificial Sequence
  Synthetic construct
gaagatgaagacagatggtgcagccaccgtacgtttgatctccagcttggtcc

SEQ ID NO: 50 - DNA, 53 bp, Artificial Sequence
  Synthetic construct
gaagatgaagacagatggtgcagccaccgtacgtttgatmtccacyttggtcc

SEQ ID NO: 51 - DNA, 53 bp, Artificial Sequence
  Synthetic construct
gaagatgaagacagatggtgcagccaccgtacgtttaatctccagtcgtgtcc

SEQ ID NO: 52 - DNA, 66 bp, Artificial Sequence
  Synthetic construct
cttcttgcatctatgttcgttttttctattgctacaaacgcgtatgctgaggtgcagctggtggag

SEQ ID NO: 53 - DNA, 66 bp, Artificial Sequence
  Synthetic construct
cttcttgcatctatgttcgttttttctattgctacaaacgcgtatgctcaggtgcagctggtggag

SEQ ID NO: 54 - DNA, 34 bp, Artificial Sequence
  Synthetic construct
ggccttttgtgctagctgaggagacrgtgaccag

SEQ ID NO: 55 - DNA, 34 bp, Artificial Sequence
  Synthetic construct
ggccttttgtgctagctgaagagacggtgaccat

SEQ ID NO: 56 - DNA, 34 bp, Artificial Sequence
  Synthetic construct
ggccttttgtgctagctgaggagacggtgaccag

SEQ ID NO: 57 - DNA, 34 bp, Artificial Sequence
  Synthetic construct
ggccttttgtgctagctgaggagacggtgaccgt

SEQ ID NO: 58 - DNA, 541 bp, Artificial Sequence
  Synthetic construct
cgtacggtggctgcaccatctgtcttcatcttcccgccatctgatgaacagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgaaaaacataaagtctacgcctgcgaagtcacccatcagggcctgagctcgcccgtcacaaagagcttcaacaggggagagtgtggtggttctgattacaaagatgacgatgacaaataattaactcgaggctgagcaaagcagactactaataacataaagtctacgccggacgcatcgtggccctagtacgcaagttcacgtaaaaagggtaactagaggttgaggtgattttatgaaaaagaatatcgcatttcttcttgcatctatgttcgttttttctattgctacaaacgcgtatgct

SEQ ID NO: 59 - protein, 107 aa, Artificial Sequence
  Synthetic construct
AIRMTQSPSSLSASVGDRVTITCRASQDISSWLAWYQQKPDKAPKSLIYAASSLEDGVPSRFSGSGSGTDFTLTISSLQAEDFATYYCQQYNTYPWTFGQGTKVEIK

SEQ ID NO: 60 - protein, 119 aa, Artificial Sequence
  Synthetic construct
EVQLVESGGGVVQPGGSLRLSCAASGFTFSSYAMHWVRQAPGKGLEWVAVISYDGSNKYYADSVKGRFTISRDNSKNTLYLQMNSLRAEDTAVYYCARDDRFFGGMDVWGQGTTVTVSS

SEQ ID NO: 61 - protein, 107 aa, Artificial Sequence
  Synthetic construct
DIQMTQSPSSLSASVGDRVTITCRASQLVDTAVAWYQQKPGKAPKLLIYFASYLYSGVPSRFSGSRSGTDFTLTISSLQPEDFATYYCQQVVYYPPTFGQGTKVEIK

SEQ ID NO: 62 - protein, 118 aa, Artificial Sequence
  Synthetic construct
EVQLVESGGGLVQPGGSLRLSCAASGFNIKYTYIHWVRQAPGKGLEWVALIDPHYGFTRYADSVKGRFTISADTSKNTAYLQMNSLRAEDTAVYYCSRWYYDHAMDYWGQGTLVTVSS

SEQ ID NO: 63 - protein, 110 aa, Artificial Sequence
  Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQAYFFHHFPPTFGQGTKVEIK

SEQ ID NO: 64 - protein, 120 aa, Artificial Sequence
  Synthetic construct
EVQLVESGGGLVQPGGSLRLSCAASGFNIKDTYIHWVRQAPGKGLEWVAFIDPIFGDTRYADSVKGRFTISADTSKNTAYLQMNSLRAEDTAVYYCSRWGGDGFYAMDYWGQGTLVTVSS

SEQ ID NO: 65 - protein, 214 aa, Artificial Sequence
  Synthetic construct
AIRMTQSPSSLSASVGDRVTITCRASQDISSWLAWYQQKPDKAPKSLIYAASSLEDGVPSRFSGSGSGTDFTLTISSLQAEDFATYYCQQYNTYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 449 aa, Artificial Sequence
  Synthetic construct
EVQLVESGGGVVQPGGSLRLSCAASGFTFSSYAMHWVRQAPGKGLEWVAVISYDGSNKYYADSVKGRFTISRDNSKNTLYLQMNSLRAEDTAVYYCARDDRFFGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 214 aa, Artificial Sequence
  Synthetic construct
DIQMTQSPSSLSASVGDRVTITCRASQLVDTAVAWYQQKPGKAPKLLIYFASYLYSGVPSRFSGSRSGTDFTLTISSLQPEDFATYYCQQVVYYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 448 aa, Artificial Sequence
  Synthetic construct
EVQLVESGGGLVQPGGSLRLSCAASGFNIKYTYIHWVRQAPGKGLEWVALIDPHYGFTRYADSVKGRFTISADTSKNTAYLQMNSLRAEDTAVYYCSRWYYDHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 217 aa, Artificial Sequence
  Synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQAYFFHHFPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 450 aa, Artificial Sequence
  Synthetic construct
EVQLVESGGGLVQPGGSLRLSCAASGFNIKDTYIHWVRQAPGKGLEWVAFIDPIFGDTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 11 aa, Artificial Sequence
  Synthetic construct
RASQDISSWLA

SEQ ID NO: 72 - protein, 7 aa, Artificial Sequence
  Synthetic construct
AASSLED

SEQ ID NO: 73 - protein, 9 aa, Artificial Sequence
  Synthetic construct
QQYNTYPWT

SEQ ID NO: 74 - protein, 5 aa, Artificial Sequence
  Synthetic construct
SYAMH

SEQ ID NO: 75 - protein, 17 aa, Artificial Sequence
  Synthetic construct
VISYDGSNKYYADSVKG

SEQ ID NO: 76 - protein, 10 aa, Artificial Sequence
  Synthetic construct
DDRFFGGMDV

SEQ ID NO: 77 - protein, 11 aa, Artificial Sequence
  Synthetic construct
RASQLVDTAVA

SEQ ID NO: 78 - protein, 7 aa, Artificial Sequence
  Synthetic construct
FASYLYS

SEQ ID NO: 79 - protein, 9 aa, Artificial Sequence
  Synthetic construct
QQVVYYPPT

SEQ ID NO: 80 - protein, 5 aa, Artificial Sequence
  Synthetic construct
YTYIH

SEQ ID NO: 81 - protein, 17 aa, Artificial Sequence
  Synthetic construct
LIDPHYGFTRYADSVKG

SEQ ID NO: 82 - protein, 11 aa, Artificial Sequence
  Synthetic construct
SRWYYDHAMDY

SEQ ID NO: 83 - protein, 11 aa, Artificial Sequence
  Synthetic construct
RASQDVNTAVA

SEQ ID NO: 84 - protein, 7 aa, Artificial Sequence
  Synthetic construct
SASFLYS

SEQ ID NO: 85 - protein, 12 aa, Artificial Sequence
  Synthetic construct
QQAYFFHHFPPT

SEQ ID NO: 86 - protein, 5 aa, Artificial Sequence
  Synthetic construct
DTYIH

SEQ ID NO: 87 - protein, 17 aa, Artificial Sequence
  Synthetic construct
FIDPIFGDTRYADSVKG

SEQ ID NO: 88 - protein, 13 aa, Artificial Sequence
  Synthetic construct
SRWGGDGFYAMDY

SEQ ID NO: 89 - protein, 479 aa, Homo sapiens
NEHDDCQVTNPSTGHLFDLSSLSGRAGFTAAYSEKGLVYMSICGENENCPPGVGACFGQTRISVGKANKRLRYVDQVLQLVYKDGSPCPSKSGLSYKSVISFVCRPEAGPTNRPMLISLDKQTCTLFFSWHTPLACEQATECSVRNGSSIVDLSPLIHRTGGYEAYDESEDDASDTNPDFYINICQPLNPMHGVPCPAGAAVCKVPIDGPPIDIGRVAGPPILNPIANEIYLNFESSTPCLADKHFNYTSLIAFHCKRGVSMGTPKLLRTSECDFVFEWETPVVCPDEVRMDGCTLTDEQLLYSFNLSSLSTSTFKVTRDSRTYSVGVCTFAVGPEQGGCKDGGVCLLSGTKGASFGRLQSMKLDYRHQDEAVVLSYVNGDRCPPETDDGVPCVFPFIFNGKSYEECIIESRAKLWCSTTADYDRDHEWGFCRHSNSYRTSSIIFKCDEDEDIGRPQVFSEVRGCDVTFEWKTKVVCPP

SEQ ID NO: 90 - protein, 479 aa, Murine
NTHDDCQVTNPSTGHLFDLSSLSGRAGINASYSEKGLVFMSICEENENCGPGVGACFGQTRISVGKASKRLSYKDQVLQLVYENGSPCPSLSDLRYKSVISFVCRPEAGPTNRPMLISLDKQTCTLFFSWHTPLACEQATECTVRNGSSIIDLSPLIHRTGGYEAYDESEDDTSDTTPDFYINICQPLNPMHGVPCPAGASVCKVPVDGPPIDIGRVTGPPIFNPVANEVYLNFESSTHCLADRYMNYTSLITFHCKRGVSMGTPKLIRTNDCDFVFEWETPIVCPDEVKTQGCAVTDEQLLYSFNLTSLSTSTFKVTRDARTYSIGVCTAAAGLGQEGCKDGGVCLLSGNKGASFGRLASMQLDYRHQDEAVILSYVNGDPCPPETDDGEPCVFPFIYKGKSYDECVLEGRAKLWCSKTANYDRDHEWGFCRQTNSYRMSAIIFTCDESEDIGRPQVFSEDRGCEVTFEWKTKVVCPP

SEQ ID NO: 91 - protein, 479 aa, Canis familiaris
NVHDNCQVTNPATGHLFDLSSLSGRAGHTAAYSEKGLVYISICEDNENCSPGVGACFGQTRISVGKANKRLTYVDQVLQLVYEDGSPCPSKSGLTYKSVISFVCRPEAGPTNRPMLISLDKQTCTLFFSWHTPLACEQVTECSVRNGSSIIDLSPLIHRTGGYEAYDESEDDTSDTGPDFYINICQPLNPMHGVPCPAGAAVCKVPVDGPPIDIGRVTGPPILNPIANEVYLNFESSTPCLADKHFNYTSLIAFHCRRGVSMGAPMLLRTSDCDFVFGWETPLVCPDEVKMDGCSLTDEQLHYSFNLSSLSKSTFKVTRDSRTYSIGVCTAAAGLDEGGCKDGGVCLLSGSKGASFGRLASMRLDYRHQDEAVILSYANGDNCPPETEAGDPCVFPFIFNGKSYEECVVEGRARLWCSTTANYDRDHEWGFCRHSNSHRMSSIIFKCDEDADIGRPQVFSEVRGCEVTFEWKTKVVCPP

(19) United States
(12) Patent Application Publication
(10) Pub. No.: US 2024/0124595 A1
(43) Pub. Date: Apr. 18, 2024

(54) ANTIBODIES TO IGF2R AND METHODS

(71) Applicant: UNIVERSITY OF SASKATCHEWAN, Saskatoon (CA)

(72) Inventors: Maruti Uppalapati, Saskatoon (CA); Ekaterina Dadachova, Saskatoon (CA)

(21) Appl. No.: 18/277,831

(22) PCT Filed: Feb. 25, 2022
(86) PCT No.: PCT/CA2022/050273

§ 371 Date: Aug. 18, 2023

Related U.S. Application Data
(60) Provisional application No. 63/153,756, filed on Feb. 25, 2021.
(60) Provisional application No. 63/291,773, filed on Dec. 20, 2021.

(51) Int. Cl. C07K 16/28; A61K 51/10; A61P 35/00; G01N 33/574

(52) U.S. Cl. CPC C07K 16/2863; A61K 51/103; A61P 35/00; G01N 33/57407; G01N 33/57492; A61K 2039/505; C07K 2317/33; C07K 2317/34; C07K 2317/92

(57) ABSTRACT

The disclosure relates to antibodies that bind human, murine, and canine IGF2R, and methods of using said antibodies. Provided herein are antibodies having specific CDRs identified herein, including functional variants of specific variable domains and IgGs having the specified CDR sequences, and immunoconjugates of said antibodies and uses thereof. Also provided herein are compositions and kits comprising said antibodies, and methods and uses of said antibodies, immunoconjugates, compositions, and kits. Also provided herein are the use of said antibodies for radioimmunotherapy (RIT) for cancer including osteosarcoma.

### CROSS-REFERENCE TO RELATED APPLICATIONS

This application claims the benefit of priority to U.S. Provisional Application Nos. 63/153,756, filed Feb. 25, 2021 and 63/291,773, filed Dec. 20, 2021, the contents of each of which are incorporated herein by reference in their entirety.

### INCORPORATION OF SEQUENCE LISTING

A computer readable form of the Sequence Listing “P63822PC00_ST25_ Sequence_Listing” (62,420 bytes) created on Feb. 24, 2022, is herein incorporated by reference.

### FIELD

The present disclosure relates to the development of antibodies to Insulin Growth Receptor Type 2 (IGF2R) and the use of these antibodies for radio-imaging and treatment of IGF2R-expressing cancer such as osteosarcoma and in particular, canine and human osteosarcoma.

### INTRODUCTION

Osteosarcoma (OS) is the most common primary malignant bone tumor and the fifth most common primary malignancy among adolescents and young adults (Smith et al). Unfortunately, overall survival has plateaued at approximately 70%, with no meaningful improvement realized in over 20 years (Meyers et al; Ferrari et al). Patients who demonstrate overt metastatic disease generally have poor outcomes, with metastases to the lungs and to the bone portending an overall survival of less than 40% and 20%, respectively. Unlike some cancers, which share a common genetic signature, OS demonstrates sweeping genetic variability from one tumor to the next, marked by complex karyotypes and substantial aneuploidy (Ladanyi et al). This has made pursuing a conventional targeted treatment approach challenging and has led recent cooperative efforts to consider alternate approaches that involve commonalities such as metastatic patterns and tumor microenvironment (Gorlick et al). Commonalities are becoming increasingly relevant given OS rarity, genetic variability and in some cases, resistance to conventional treatment strategies. The consistent surface overexpression of the cation independent mannose-6-phosphate/insulin-like growth factor-2 receptor (IGF2R) has been identified across multiple standard and patient-derived OS cell lines (Hassan et al). It had been shown previously that a single nucleotide polymorphism (SNP) within a haplotype block in IGF2R is associated with an increased risk of developing OS (Savage et al). In addition, IGF2R is also expressed by and plays an important role in following cancers: pleomorphic adenoma (Arslan I, et al); intestinal adenoma (Hughes J, et al); medulloblastomas (Bharambe H S, et al); adenocortical carcinoma (De Martino M C, et al); mucosal melanoma (lida Y, et al.); and hepatocellular carcinoma (Lautem A, et al.).

Targeted radionuclide therapy (TRT) delivers cytocidal radiation in form of alpha- or beta-particles emitting radionuclides to the tumor with high precision, thus avoiding a lot of side of effects of external beam radiation therapy (EBRT). Recent regulatory approvals include 223Radium chloride (Xofigo) for treatment of prostate cancer metastatic to the bone and of 177Lutetium-labeled peptide (Lutathera) for treatment of somatostatin receptor-positive gastroenteropancreatic neuroendocrine tumors (GEP-NETs). Radioimmunotherapy (RIT) is a subset of TRT and is a method of delivering cytotoxic radiation in a targeted fashion whereby an antigen-specific antibody is bound to either an alpha- or beta-emitting radioisotope (Milenic et al; Larson et al). RIT was regulatory approved more than a decade ago for refractory and recurrent non-Hodgkin's lymphoma (Zevalin) (Kaminski et al).

Several strategies have been tried for therapy of OS. Immunotherapy with unlabeled mAbs has been evaluated in clinical trials using immune check-point inhibitors (anti-CTLA4, and anti-PD-1) with no appreciable success (Wedekind et al). Larsen et al. performed in vitro evaluation of 211Astatine-labeled TP-3 mAb for killing of OS cells (Larsen et al). This antibody was raised against osteosarcoma-associated cell membrane antigen and is believed to bind to an epitope on an alkaline phosphatase isoform expressed on both canine and human osteosarcoma cells (Bruland et al.).

Recently the same group armed anti-CD146 mAb with 177Lutetium and reported biodistribution and dosimetry results in a mouse model of OS (Westrom et al). CD146 is normally expressed by vascular endothelial cells, smooth muscle cells, and pericytes, making CD146 antibodies unsuitable for RIT.

Previous work has demonstrated the preferential tumor localization of the radiolabeled mouse monoclonal antibodies (mAbs) to human IGF2R (mAb MEM-238) and to human and murine IGF2R (mAb 2G11) in OS xenografts and patient-derived xenografts (PDX) in mice in comparison with the control mAbs. Treatment of OS tumors using 188Rhenium (¹⁸⁸Re) and 177Lutetium (¹⁷⁷Lu)-labeled IGF2R-specific mAbs MEM-238 and 2G11 resulted in tumor growth inhibition and possibly regression while being safe to normal organs in mice (Geller et al; Karkare et al). mAb 2G11 bound specifically to IGF2R on the tumors from two randomly selected cases of canine OS as demonstrated by immunohistochemistry (IHC) (Karkare et al).

Cancer has become a leading cause of death in companion animals now that more pets are living long enough to develop the disease. Furthermore, more owners are seeking advanced and novel therapies for their pets. Living in the same environments, pets and humans are often afflicted by the same types of cancer which show similar behavior and, in some species, express the same antigens (Riccardo et al). Canine OS exhibits similar to human OS clinical presentations and molecular aberrations. Annually 10,000 new canine cases are diagnosed cases in the United States alone. 90% of canine patients will die from metastasis within 1 year of diagnosis. In this regard, canine and human OS shares certain antigens and can be targeted with the same mAbs (Haines and Bruland) and the mAbs to human cation independent mannose-6-phosphate receptor also bind to canine one (Prydz et al).

Treatments and diagnostic agents for cancers such as osteosarcoma are desirable for humans and/or companion animals.

### SUMMARY

As demonstrated herein, the inventors have developed pan-antibodies to human, canine and murine IGF2R and demonstrated their in vitro and in vivo use as agents for radioimmunoimaging of canine and human OS and in vivo use for radioimmunotherapy (RIT) in canine OS-bearing mice. The antibodies described herein can be used for radioimmunotherapy (RIT) of human and/or canine cancers that express IGF2R such as pleomorphic adenoma, intestinal adenoma, medulloblastoma, adenocortical carcinoma, mucosal melanoma, hepatocellular carcinoma, or osteosarcoma (OS).

Described also herein is the evaluation of binding of antibodies and binding fragments thereof to IGF2R. These antibodies can be used for radioimmunoimaging and RIT of OS or other cancers that express IGF2R. They can be used for example in methods involving adults, children, adolescents, or companion animals such as dogs.

An aspect includes an antibody which specifically binds an epitope in domains 11-13 of human, murine, and canine insulin-like growth factor-2 receptor (IGF2R), domains 11-13 of human, murine, and canine IGF2R having the amino acid sequence as set forth in SEQ ID NOs: 89, 90, and 91, wherein the antibody binds human, murine, and/or canine IGF2R with at least or about 2-fold, at least or about 3-fold, at least or about 4-fold, or at least or about 5-fold greater affinity than monoclonal mouse antibody 2G11 as determined by flow cytometry using OS cells selected from OS33 cells, McKinley cells, and Gracie cells.

In an embodiment, the antibody comprises a light chain variable region and a heavy chain variable region, the light chain variable region comprising complementarity determining regions CDR-L1, CDR-L2, and CDR-L3, and the heavy chain variable region comprising complementarity determining regions CDR-H1, CDR-H2, and CDR-H3, wherein the amino acid sequences of said CDRs are:

| | CDR-L1 |
|---|---|
| (SEQ ID NO: 71) | |
| | RASQDISSWLA; |
| | CDR-L2 |
| (SEQ ID NO: 72) | |
| | AASSLED; |
| | CDR-L3 |
| (SEQ ID NO: 73) | |
| | QQYNTYPWT; |
| | CDR-H1 |
| (SEQ ID NO: 74) | |
| | SYAMH; |
| | CDR-H2 |
| (SEQ ID NO: 75) | |
| | VISYDGSNKYYADSVKG; |
| | and |
| | CDR-H3 |
| (SEQ ID NO: 76) | |
| | DDRFFGGMDV; |
| | CDR-L1 |
| (SEQ ID NO: 77) | |
| | RASQLVDTAVA; |
| | CDR-L2 |
| (SEQ ID NO: 78) | |
| | FASYLYS; |
| | CDR-L3 |
| (SEQ ID NO: 79) | |
| | QQVVYYPPT; |
| | CDR-H1 |
| (SEQ ID NO: 80) | |
| | YTYIH; |
| | CDR-H2 |
| (SEQ ID NO: 81) | |
| | LIDPHYGFTRYADSVKG; |
| | and |
| | CDR-H3 |
| (SEQ ID NO: 82) | |
| | SRWYYDHAMDY; |
| | or |
| | CDR-L1 |
| (SEQ ID NO: 83) | |
| | RASQDVNTAVA; |
| | CDR-L2 |
| (SEQ ID NO: 84) | |
| | SASFLYS; |
| | CDR-L3 |
| (SEQ ID NO: 85) | |
| | QQAYFFHHFPPT; |
| | CDR-H1 |
| (SEQ ID NO: 86) | |
| | DTYIH; |
| | CDR-H2 |
| (SEQ ID NO: 87) | |
| | FIDPIFGDTRYADSVKG; |
| | and |
| | CDR-H3 |
| (SEQ ID NO: 88) | |
| | SRWGGDGFYAMDY. |

In an embodiment, the light chain variable region and heavy chain variable region comprise i) a polypeptide having an amino acid sequence of SEQ ID NOs: 59 and 60; SEQ ID NOs: 61 and 62; or SEQ ID NOs: 63 and 64; ii) a polypeptide having an amino acid sequence with at least 80%, at least 90%, or at least 95% sequence identity to SEQ ID NOs: 59 and 60; SEQ ID NOs: 61 and 62; or SEQ ID NOs: 63 and 64 wherein the CDR sequences are those shown underlined therein; or iii) a conservatively substituted amino acid sequence of i) wherein the CDR sequences are those shown underlined therein.

In an embodiment, the antibody is a humanized or human antibody.

In an embodiment, the antibody is a single chain antibody.

In an embodiment, the antibody is an antibody fragment selected from Fab, Fab′, F(ab′)2, scFv, dsFv, ds-scFv, dimers, nanobodies, minibodies, diabodies, and multimers thereof.

In an embodiment, the antibody is an IgG, optionally IgG1.

In an embodiment, the antibody comprises a light chain and a heavy chain the light chain and heavy chain comprising i) a polypeptide having an amino acid sequence of SEQ ID NO: 65 and 66, SEQ ID NO: 67 and 68, or SEQ ID NO: 69 and 70 respectively; ii) a polypeptide having an amino acid sequence with at least 80%, at least 90%, or at least 95% sequence identity to SEQ ID NO: 65 and 66, SEQ ID NO: 67 and 68, or SEQ ID NO: 69 and 70 wherein the CDR sequences are those of SEQ ID NOs: 71-75, SEQ ID NOs: 77-82, or SEQ ID NOs: 83-88; or iii) a conservatively substituted amino acid sequence of i) wherein the CDR sequences are those of SEQ ID NOs: 71-75, SEQ ID NOs: 77-82, or SEQ ID NOs: 83-88.

In an embodiment, the light chain comprises a polypeptide having an amino acid sequence of SEQ ID NO: 69 and the heavy chain comprise a polypeptide having an amino acid sequence of SEQ ID NO: 70.

In an embodiment, the antibody competes for binding to domains 11-13 of human, murine, or canine insulin-like growth factor-2 receptor (IGF2R) with an antibody described herein.

An aspect includes a nucleic acid molecule encoding an antibody described herein.

An aspect includes a vector comprising a nucleic acid molecule encoding an antibody described herein.

An aspect includes a cell comprising a nucleic acid molecule or vector encoding an antibody described herein, or expressing an antibody described herein.

An aspect includes an immunoconjugate comprising an antibody described herein and a therapeutic agent, or detectable label.

In an embodiment, the detectable label or therapeutic agent is a radionuclide, optionally an alpha- beta- or gamma-emitting radionuclide.

In an embodiment, the antibody is labeled with the radionuclide using a bifunctional linker, optionally CHXA″.

In an embodiment, the radionuclide is selected from 223Radium, 177Lutetium, 188Rhenium, 111Indium, and 225Actinium.

In an embodiment, the light chain comprises a polypeptide having an amino acid sequence of SEQ ID NO: 69, the heavy chain comprise a polypeptide having an amino acid sequence of SEQ ID NO: 70, and the radionuclide is 111Indium or 177Lutetium.

An aspect includes a composition comprising an antibody, nucleic acid molecule, vector, cell, or immunoconjugate described herein, and a diluent or pharmaceutically acceptable carrier.

An aspect includes an antibody or composition described herein for use in treating cancer, optionally the cancer is selected from osteosarcoma, pleomorphic adenoma, intestinal adenoma, medulloblastoma, adrenocortical carcinoma, mucosal melanoma, and hepatocellular carcinoma, preferably the cancer is osteosarcoma, in a subject in need thereof.

In an embodiment, the subject is a human or a canine.

An aspect includes a method for detecting IGF2R expression in a biological sample, the method comprising a) obtaining a biological sample suspected of containing IGF2R, b) contacting the sample with an antibody or immunoconjugate described herein under conditions permissive for forming an antibody:IGF2R complex, and c) detecting the presence of any complex, wherein the presence of detectable complex is indicative that the sample expresses IGF2R.

An aspect includes a method of detecting whether a subject has an IGF2R-expressing cancer, the method comprising obtaining a biological sample suspected of containing an IGF2R-expressing cancer cell from the subject, contacting the sample with an antibody or immunoconjugate described herein under conditions permissive for forming an antibody:IGF2R complex, and detecting the presence of an antibody complex, wherein the presence of an antibody complex indicates that the subject has an IGF2R-expressing cancer, optionally the IGF2R-expressing cancer is selected from osteosarcoma, pleomorphic adenoma, intestinal adenoma, medulloblastoma, adenocortical carcinoma, mucosal melanoma, and hepatocellular carcinoma, optionally the IGF2R-expressing cancer is osteosarcoma.

In an embodiment, the biological sample is obtained from a subject having or suspected of having a cancer, optionally osteosarcoma, optionally the biological sample is a tumor sample.

An aspect includes a method for imaging an IGF2R-expressing tumor in a subject, the method comprising administering an antibody, immunoconjugate, or composition described herein to the subject, and detecting the presence of the label, optionally the antibody is an IgG.

An aspect includes a method of determining if a subject has an IGF2R-expressing tumor, the method comprising administering an antibody, immunoconjugate, or composition described herein to the subject, and detecting the presence of the label by imaging, optionally the subject has or is suspected of having an IGF2R-expressing cancer, optionally the IGF2R-expressing cancer is selected from osteosarcoma, pleomorphic adenoma, intestinal adenoma, medulloblastoma, adenocortical carcinoma, mucosal melanoma, and hepatocellular carcinoma, preferably the IGF2R-expressing cancer is osteosarcoma. In an embodiment, the subject is a human or a canine. In an embodiment, the light chain comprises a polypeptide having an amino acid sequence of SEQ ID NO: 69, the heavy chain comprise a polypeptide having an amino acid sequence of SEQ ID NO: 70, and the radionuclide is 111Indium or 177Lutetium.

An aspect includes a method of treating a cancer, optionally the cancer is selected from osteosarcoma, pleomorphic adenoma, intestinal adenoma, medulloblastoma, adenocortical carcinoma, mucosal melanoma, and hepatocellular carcinoma, preferably the cancer is osteosarcoma, in a subject in need thereof, the method comprising administering an effective amount of an antibody, immunoconjugate, or composition described herein to the subject, preferably the antibody is an IgG. In an embodiment, the subject is a human or a canine. In an embodiment, the light chain comprises a polypeptide having an amino acid sequence of SEQ ID NO: 69, the heavy chain comprise a polypeptide having an amino acid sequence of SEQ ID NO: 70, and the radionuclide is 111Indium or 177Lutetium.

In an embodiment, the method further comprises a) detecting IGF2R expression in a biological sample according to the method of any one of claims 22 to 24, wherein the biological sample is obtained from the subject, and/or b) imaging an IGF2R-expressing tumor in the subject according to the method of claim 25 or claim 26, wherein the detecting and/or imaging is done before, during, or following administering the antibody, immunoconjugate, or composition.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DETAILED DESCRIPTION OF THE DRAWINGS

Further objects, features and advantages of the disclosure will become apparent from the following detailed description taken in conjunction with the accompanying figures showing illustrative embodiments of the disclosure, in which:

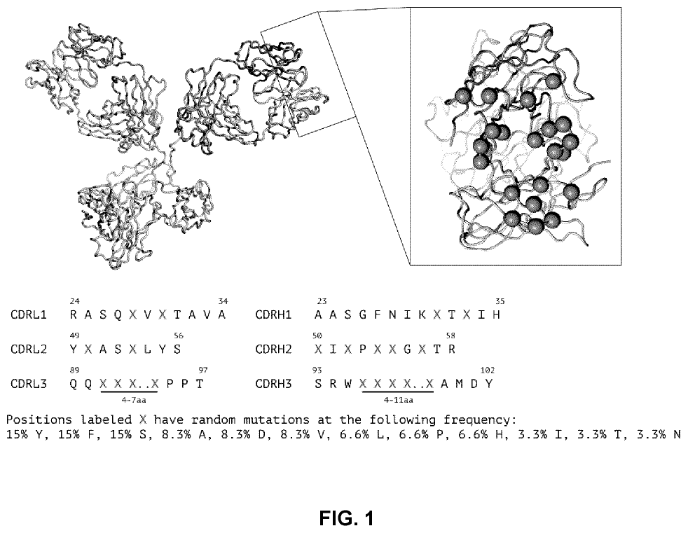

FIG. 1 shows a schematic of the design of the synthetic antibody library based on the 4D5-8 framework.

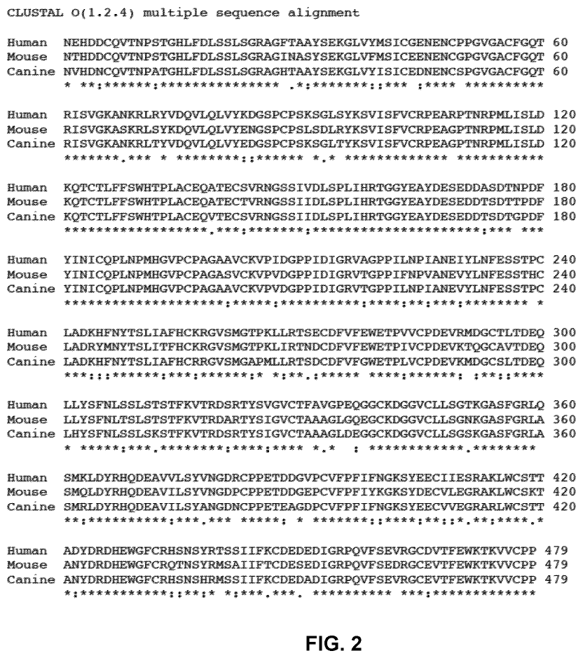

FIG. 2 shows a sequence alignment of domains 11-13 of IGF2R of human, mouse and canine origin (SEQ ID NOs: 89-91, respectively).

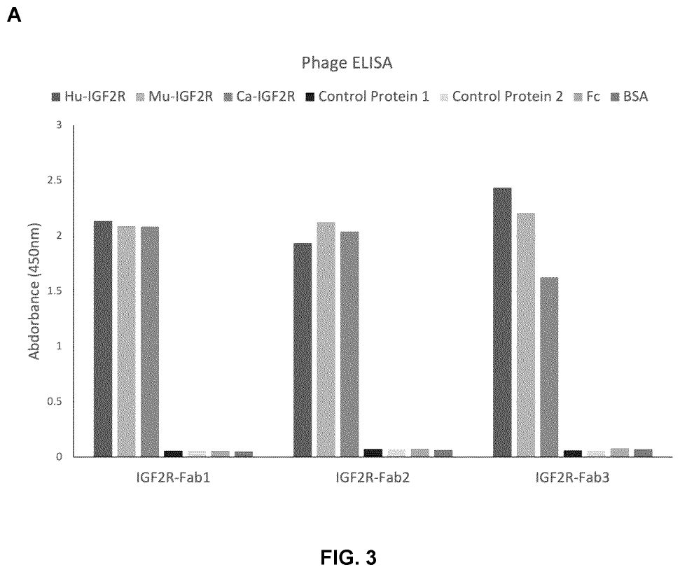

FIG. 3 represents the results of phage ELISA and competitive ELISA. A) IGF2R Fabs bind specifically to recombinant IGF2R proteins but not irrelevant control proteins. B) Binding is inhibited by competition with corresponding protein in solution at low nanomolar concentrations indicating tight binding.

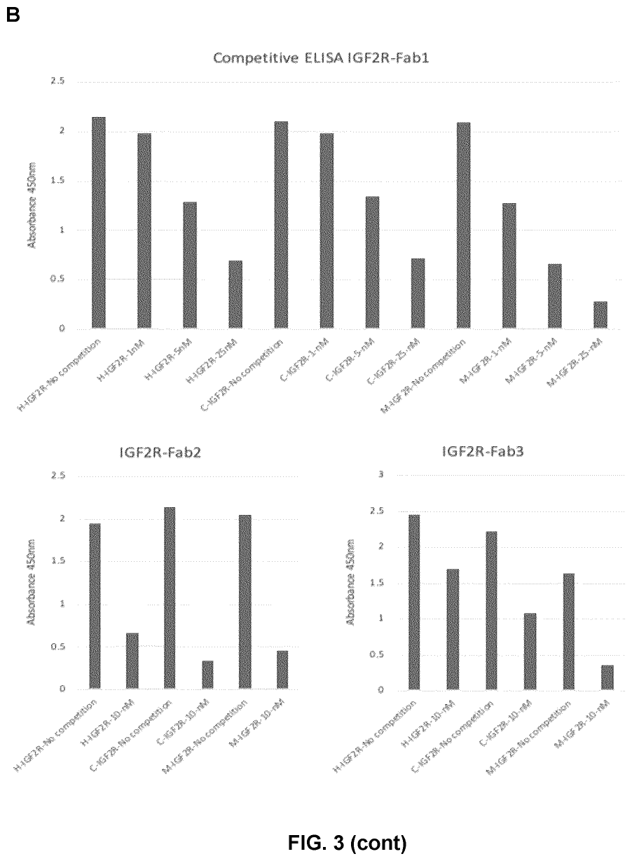

FIG. 4 shows the results of in vitro cytotoxicity of 225Ac-labeled Fab1 towards OS cell lines. Panel A shows IGF2R-positive human 143B cells; panel B shows IGF2R-negative murine K7M2 cells.

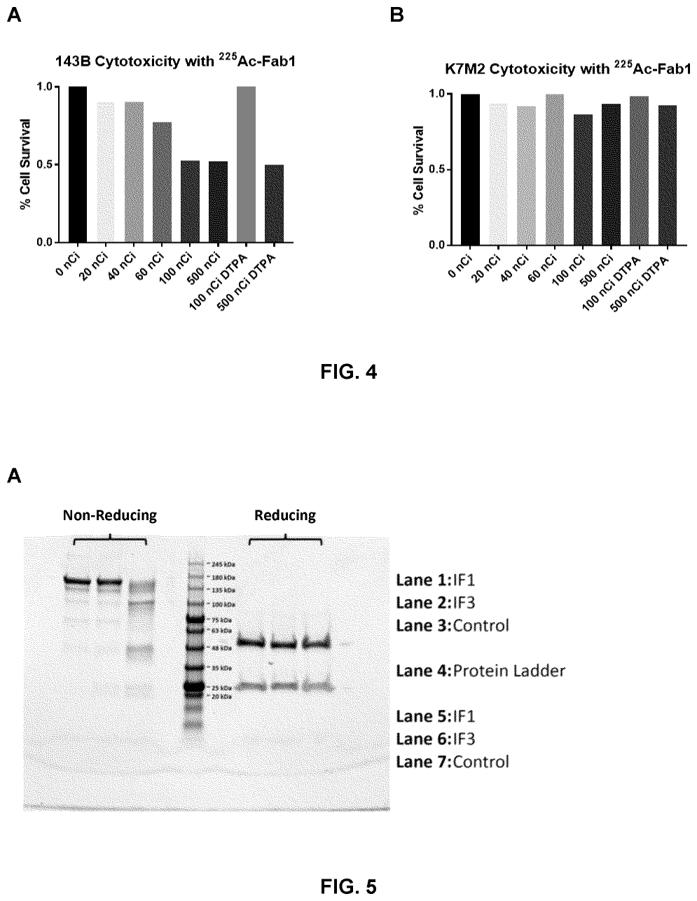

FIG. 5 shows the characterization of full length human IgGs IF1 and IF3. A, show SDS-PAGE analysis; B, C show ELISA for binding to human, canine and murine IGF2R.

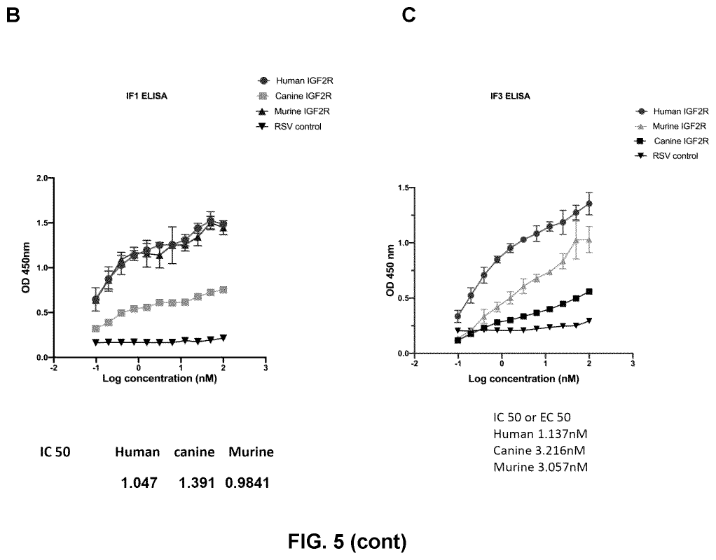

FIG. 6 shows flow cytometry results demonstrating binding of IF1 and IF3 mAbs to human and canine OS cell lines by flow cytometry. Panel A shows results for IF1, panel B shows results for IF3. Commercial murine mAb 2G11 to human and murine IGF2R was used as a positive control.

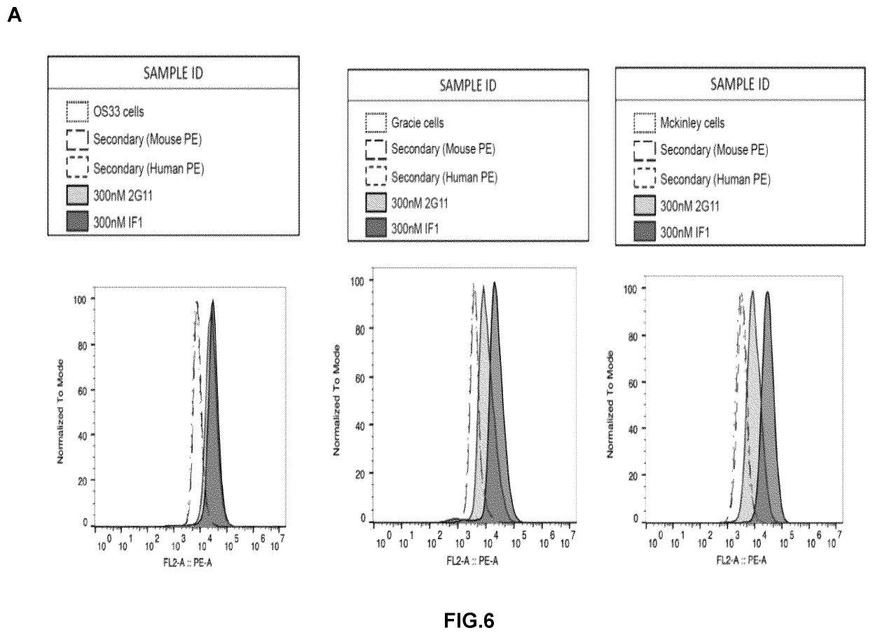

FIG. 7 shows images of immunohistochemistry of IF1 binding to randomly selected canine OS tumors. Human antibody palivizumab to respiratory syncytial virus (RSV) was used as a negative control (right panels). Original magnification 400X.

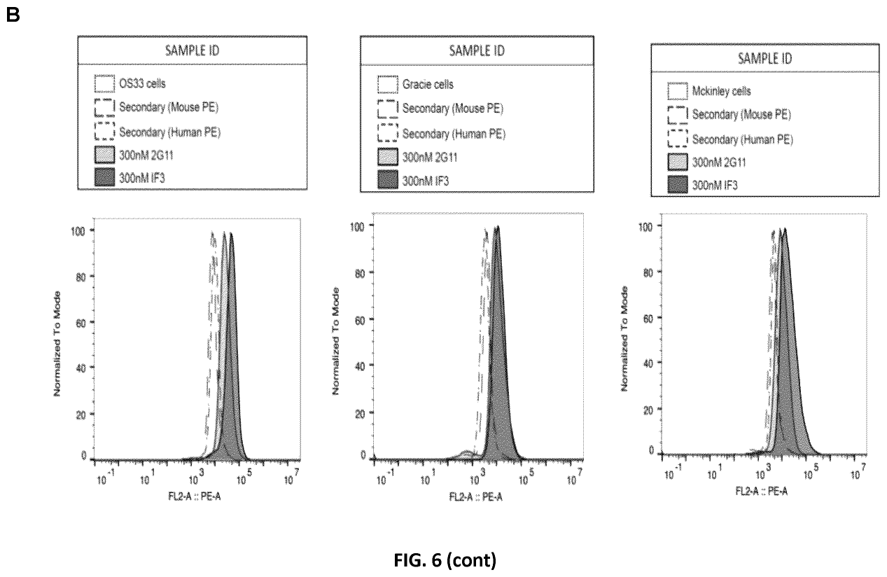

FIG. 8 is a bar graph showing the results of an IGF2R ELISA for IF1 and IF3 conjugated to 2.5 and 10 initial molar ratio of CHXA″ bifunctional linker to the antibody. Human antibody palivizumab to respiratory syncytial virus (RSV) was used as a negative control.

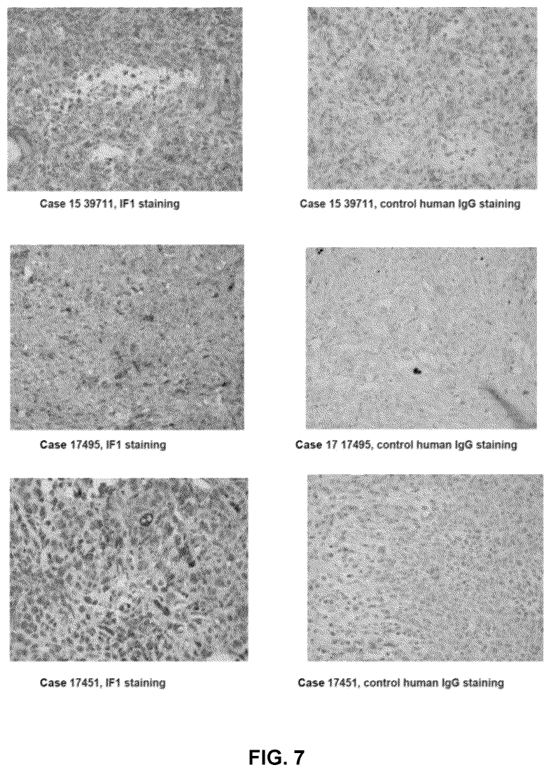

FIG. 9 is a bar graph representing the biodistribution of 111In-labeled IF1 and IF3 in female SCID mice. 2.5 and 10 initial molar ratios of CHXA″ bifunctional linker to the antibody were used.

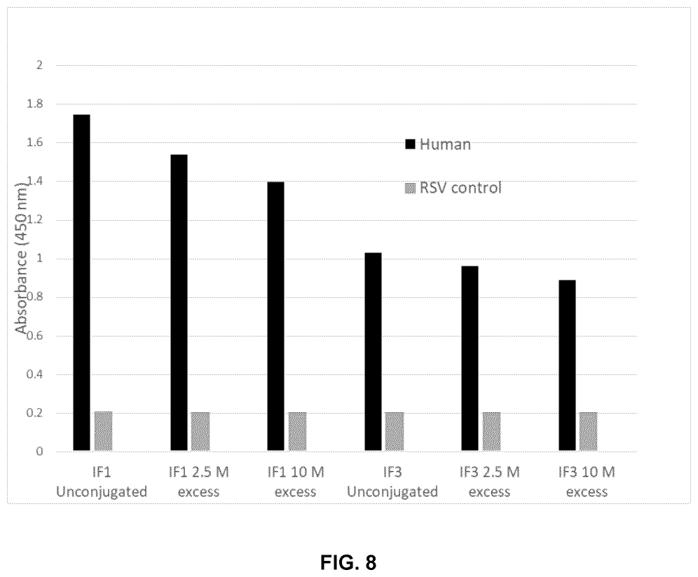

FIG. 10 shows microSPECT/CT imaging of human OS bearing mice. Female SCID mice were implanted with 143B human osteosarcoma cells and imaged with 111In-labeled IF3 on day 10 post tumor cells implantation. The tumor uptake on the image obtained 48 hrs post administration of the radiolabeled antibody in shown with a black circle.

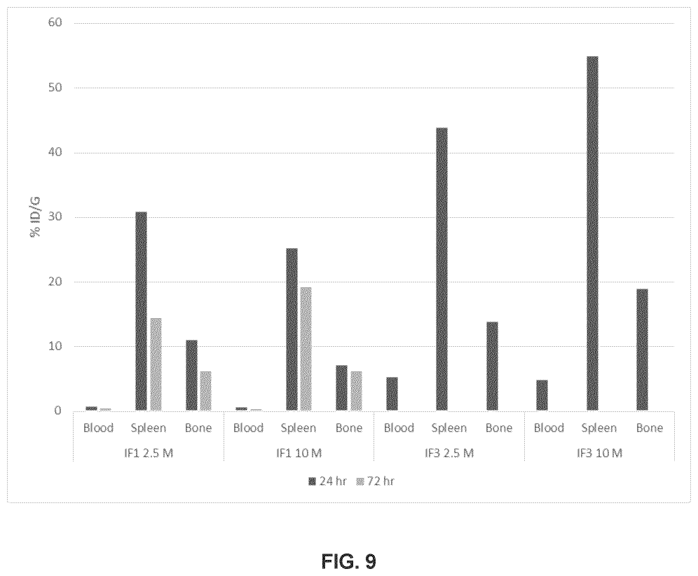

FIG. 11 is a bar graph representing the biodistribution of 111In-labelled IF3 after IP administration, at 2, 24, 48 and 72 hrs post-administration in female SCID mice bearing canine patient derived Gracie tumor.

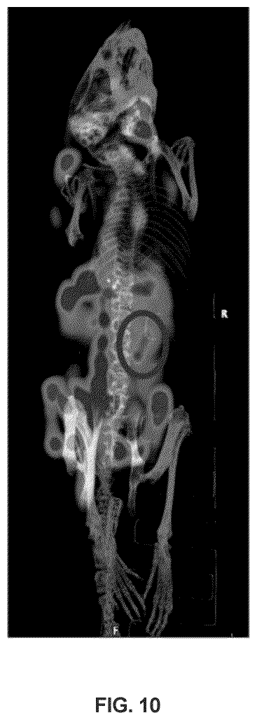

FIG. 12 shows microSPECT/CT imaging of a female SCID mouse bearing canine patient derived Gracie tumor injected IV with 111In-IF3. 24 hrs post injection is on the left, and 48 hrs post injection is on the right. The uptake in the tumor, spleen and excretion through the bladder are seen. Arrows are pointing to the tumors.

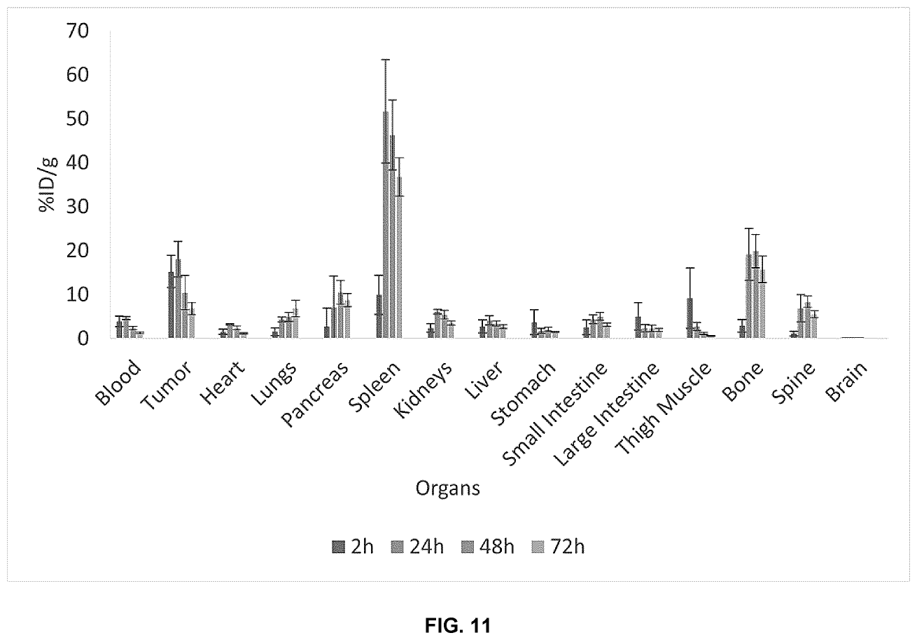

FIG. 13 shows tumor volumes in mice bearing Gracie tumors treated with 177Lu-IF3 mAb with or without pre-blocking with unlabeled (“cold”) 1F3 mAb.

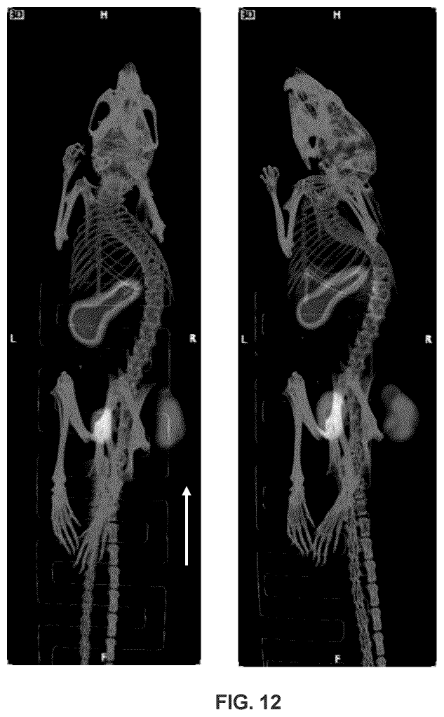

FIG. 14 shows mouse body weight in mice bearing Gracie tumors treated with 177Lu-IF3 mAb with or without pre-blocking with unlabeled (“cold”) 1F3 mAb.

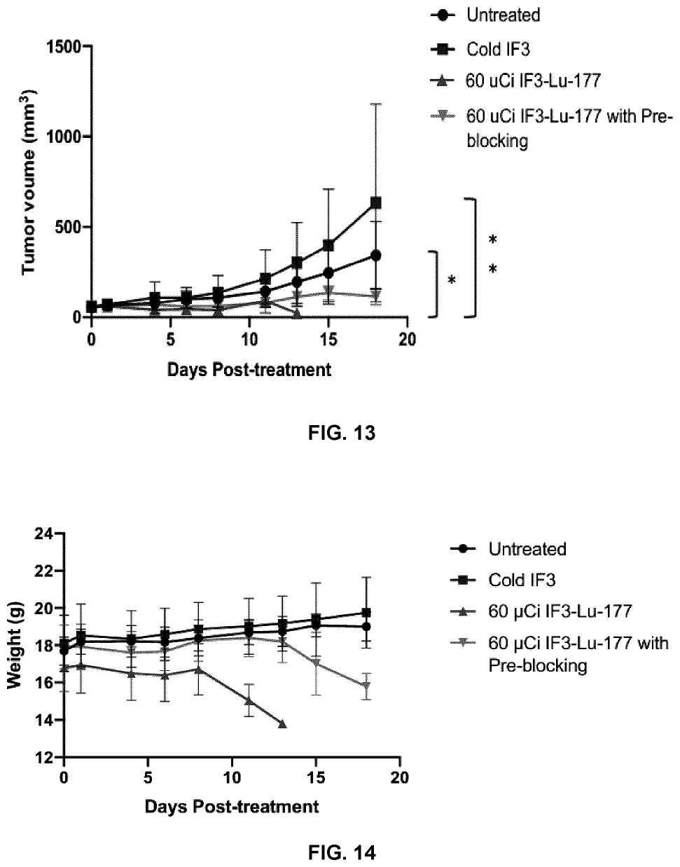

FIG. 15 shows Kaplan-Meier survival curves in mice bearing Gracie tumors treated with 177Lu-IF3 mAb with or without pre-blocking with unlabeled (“cold”) 1F3 mAb.

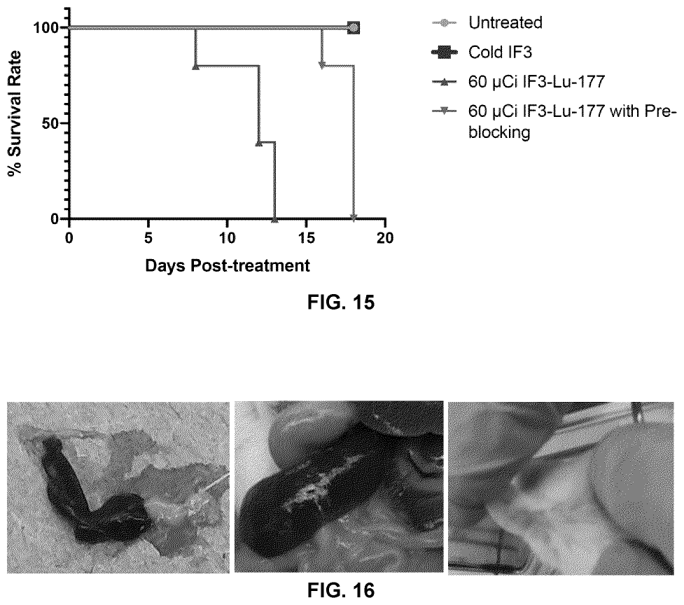

FIG. 16 shows gross pathology of the mice post-mortem. Left plate shows the spleen of a RIT alone treated mouse. The middle plate shows the spleen of an untreated mouse. The right plate shows petechiae on the ears of a RIT alone treated mouse.

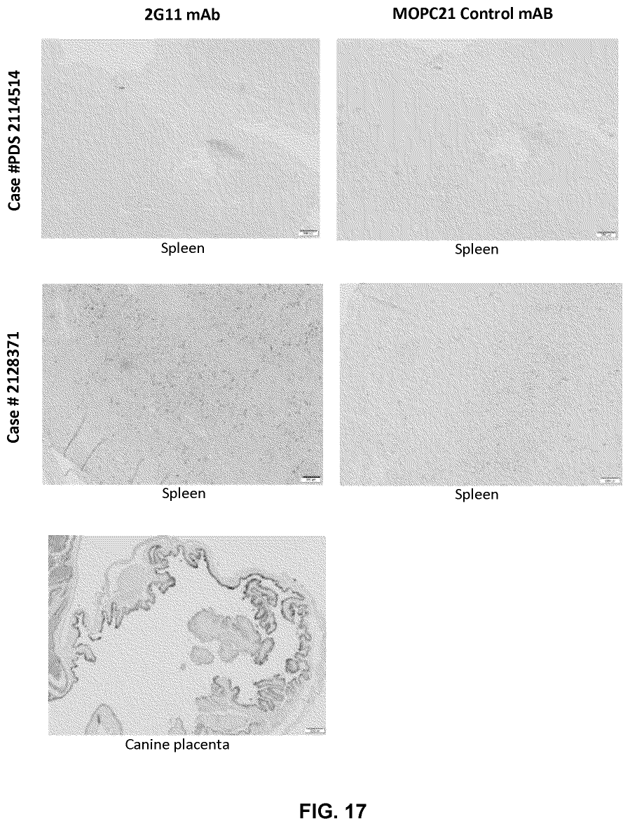

FIG. 17 shows immunohistochemistry of canine spleens with 2G11 IGF2R-specific antibody or MOPC21 control antibody. Scale bar—200 μm.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

The extracellular domain of IGF2R contains 15 cation-independent mannose receptor (CIMR) domains repeats and one FNII domain located between CIMR domains 12 and 13. IGF2R contains binding sites for IGFII and phospho-mannosyl moieties. Mannosylated proteins bind domains 3, 5 and 9 (Reddy et al.). IGFII binding site is in domain 11, with domain 13 and FNII domains assisting the binding of IGFII to IGF2R (Brown et al.). A region on IGF2R extracellular domain was selected which had i) a high-degree of conservation among humans, canines and mice and ii) availability of structural data (Brown et.al. 2008). The selected region corresponds to the IGFII binding region encompassing domains 11, 12, FnII and 13 (collectively herein “domains 11-13”) of IGF2R, spanning amino acids 1511-1989 of human IGF2R (Uniprot ID P11717). A sequence alignment of this region with mouse and canine IGF2R genes shows that this region is highly conserved across these species with 82% sequence identity (FIG. 2). These regions from human, murine and canine IGF2R were expressed as Fc fusion proteins, as bait for antibody development. This conserved region enabled generation of the antibodies that are cross-reactive.

To generate antibodies that bind to conserved regions of IGF2R, both synthetic and naïve antibody Fab-fragment libraries were developed. Trastuzumab 4D5-8 clone was used as a template and structure-guided mutagenesis was used to generate a synthetic library (FIG. 1). A conservative approach was used to mutate solvent exposed amino acids in CDRL1, CDRL2, CDRH1 and CDRH2. Solvent exposed regions of CDRL3 and CDRH3 were further mutated to vary the loop lengths to generate additional diversity. A custom codon (N1)HT that encodes for any one of 12 amino acids was used, while generating a bias for Tyr, Ser, and Phe residues. A library of 5×10⁹ variants was generated using this strategy. For generating a naïve library, primers were used to amplify the antibody repertoire from pooled human peripheral blood monocyte cDNA. This strategy focused on variable heavy chain genes from the VH3 family and variable light chain genes from kappa family, as these are the most abundant in immune repertoires. These genes were cloned into display vector pHP153 to generate a naïve library of 3×10⁸ variants. Following phage panning of both libraries, three Fabs were isolated that were cross-reactive to human, murine and canine IGF2R.

The lead antibodies were expressed as full-length IgGs in human suspension cell line Expi293F. Cross-reactivity of the antibodies was confirmed by their binding to human derived tumors in vivo, to mouse spleens and canine spontaneous tumors via IHC.

In vivo evaluation of the candidate mAbs IF1 and IF3 also revealed very fast (e.g. more than 80% clearance during the first 12 hours post-injection) clearance of 111In-labeled IF1 from the blood.

Accordingly described herein are various embodiments including but not limited to antibodies, nucleic acids encoding said antibodies and vectors comprising said nucleic acids, cells expressing said antibodies, immunoconjugates, compositions as well as methods and uses of all of the foregoing.

### I. General Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

The term “about” as used herein may be used to take into account experimental error and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### II. Antibodies, Recombinant Proteins, Nucleic Acids, Immunoconjugates and Libraries

A portion of the extracellular region of IGF2R comprising cation-independent mannose receptor (CIMR) domain repeats 11, 12, FNII, and 13 (collectively herein “domains 11-13”) was found to be highly conserved among human, murine, and canine species. As described herein, recombinant proteins comprising domains 11-13 of human, murine, and canine IGF2R (SEQ ID NOs: 89-91, respectively) and naïve and synthetic Fab libraries were used to develop antibodies which are cross-reactive for human, murine, and canine IGF2R. Accordingly, provided herein are antibodies which bind and/or are cross-reactive for domains 11-13 of human, murine, and canine IGF2R.

Said antibodies include antibodies comprising complementarity determining regions (CDRs) set out in SEQ ID NOs: 71-76, SEQ ID NOs: 77-82, and SEQ ID NOs: 83-88. The antibodies described herein may comprise light- and heavy-chain variable regions set out in SEQ ID NOs: 59 and 60; SEQ ID NOs: 61 and 62; or SEQ ID NOs: 63 and 64, or variants thereof having the CDR sequences specified herein. The antibodies described herein may comprise IgG1 light- and heavy-chains as set out in SEQ ID NO: 65 and 66, SEQ ID NO: 67 and 68, or SEQ ID NO: 69 and 70, or functional variants thereof having the CDR sequences of SEQ ID NOs: 71-76, 77-82 and 83-88.

Monoclonal mouse antibody 2G11 binds human and murine IGF2R according to the manufacturer, and canine IGF2R (Karkare S. et al 2019). This antibody can enhance the expression of IGF2R on tissues which can result in undesirable toxicity to healthy organs.

The present antibodies described herein did not enhance expression of IGF2R on tissues tested.

The basic antibody structural unit is known in the art to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. Within each of the VH and VL domains are three hypervariable regions or complementarity determining regions (CDRs, commonly denoted CDR-H1, CDR-H2, CDR-H3, CDR-L1, CDR-L2, and CDR-L3). The carboxy-terminal portions of the heavy and light chains together form a constant region primarily responsible for effector function.

The term “complementarity determining region” or “CDR” as used herein refers to particular hypervariable regions of antibodies that are commonly presumed to contribute to epitope binding. Computational methods for identifying CDR sequences include Kabat, Chothia, Martin, AHo and IMGT. The CDRs listed in the present disclosure are identified using Kabat definition. A person skilled in the art having regard to the sequences comprised herein would also be able to identify CDR sequences based on IMGT and Chothia etc. Such antibodies are similarly encompassed.

The term “antibody” as used herein is intended to encompass for example monoclonal antibodies, polyclonal antibodies, humanized (as well as canine-ized) and other chimeric antibodies, and binding fragments thereof, including for example a single chain Fab fragment, Fab′2 fragment, or single chain Fv fragment. The antibody may be from recombinant sources and/or produced in transgenic animals. Also included are human antibodies that can be produced in transgenic animals or using biochemical techniques, or can be isolated from a library such as a phage display library using for example domains 11-13 of human, murine, and canine IGF2R. Antibody backbones may comprise any suitable variable heavy chain or variable light chain sequences, including, without limitation, VH3-30, VH3-23, and/or VK1 backbone sequences. Antibodies, including humanized and/or other chimeric antibodies may include sequences from one or more than one isotype, class, or species. Antibodies may be any class of immunoglobulins including: IgG, IgM, IgD, IgA, or IgE; and any isotype thereof, including IgG1, IgG2 (e.g. IgG2a, IgG2b), IgG3 and IgG4. Further, these antibodies can be produced as antigen binding fragments such as Fab, Fab′ F(ab′)2, Fd, Fv and single domain antibody fragments, or as single chain antibodies in which the heavy and light chains are linked by a spacer. The antibodies may include sequences from any suitable species including human and canine. The antibodies may be bi-specific or multi-specific antibodies. Also, the antibodies may exist in monomeric or polymeric form. Antibodies and nucleic acids that encode them may also comprise a signal sequence moiety including for example a signal peptide from heat-stable enterotoxin II or a signal peptide from IL2. Other signal peptides and the nucleic acids that encode them are known in the art.

The phrase “isolated antibody” refers to antibody produced in vivo or in vitro that has been removed from the source that produced the antibody, for example, an animal, hybridoma or other cell line (such as recombinant insect, yeast or bacteria cells that produce antibody). The isolated antibody is optionally “purified”, which means at least: 80%, 85%, 90%, 95%, 98% or 99% purity.

The term “binding fragment” as used herein to a part or portion of an antibody or antibody chain comprising fewer amino acid residues than an intact or complete antibody or antibody chain and which binds the antigen or competes with intact antibody. Exemplary binding fragments include without limitations Fab, Fab′, F(ab′)2, scFv, dsFv, ds-scFv, dimers, nanobodies, minibodies, diabodies, and multimers thereof. Fragments can be obtained via chemical or enzymatic treatment of an intact or complete antibody or antibody chain. Fragments can also be obtained by recombinant means. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be constructed by recombinant expression techniques.

The term “epitope” as commonly used means an antibody binding site, such as a stretch of amino acids having a particular structural conformation, in an antigen that is specifically recognized by the antibody. The epitope may be a linear epitope or a conformational epitope. For example an antibody generated or selected against a recombinant protein comprising a target region (e.g. domains 11-13 of IGF2R) specifically bind a stretch of amino acids (contiguous or non-adjacent) in the target region.

The term “greater affinity” as used herein refers to a relative degree of antibody binding where an antibody X binds to target Y more strongly (Kₒₙ) and/or with a smaller dissociation constant (K_{off}) than does comparator antibody Z, and in this context antibody X has a greater affinity for target Y than Z. Likewise, the term “lesser affinity” herein refers to a degree of antibody binding where an antibody X binds to target Y less strongly and/or with a larger dissociation constant than does antibody Z, and in this context antibody X has a lesser affinity for target Y than Z. The affinity of binding between an antibody and its target antigen can be expressed quantitatively as KA equal to 1/KD where KD is equal to kₒₙ/k_{off}. As such, a greater affinity corresponds to a lower KD. The kₒₙ and k_{off} values can be measured using surface plasmon resonance (SPR) technology, for example using a Molecular Affinity Screening System (MASS-1) (Sierra Sensors GmbH, Hamburg, Germany).

Binding affinity can also be assessed using techniques such as flow cytometry, radioimmunoassay (e.g. Lindmo plot), bio-layer interferometry, isothermal calorimetry, or ELISA.

The term “functional variant” as used herein for example with respect to an antibody sequence refers to an antibody sequence that includes one or more modifications compared to a comparator of sequence disclosed herein that performs substantially the same function as the comparator molecule disclosed herein in substantially the same way. For example, the functional variant may comprise a sequence having at least 80%, at least 85%, at least 90%, at least 95%, at least 98% or at least 99% sequence identity to the comparator sequence disclosed herein. The functional variant may also comprise conservatively substituted amino acid sequences of the comparator sequence disclosed herein.

Accordingly, in an embodiment, the antibody comprises a light chain variable region and a heavy chain variable region having an amino acid sequence with at least 80%, at least 85%, at least 90%, at least 95%, at least 98% or at least 99% sequence identity to SEQ ID NOs: 59 and/or 60; SEQ ID NOs: 61 and/or 62; or SEQ ID NOs: 63 and/or 64; wherein the CDR sequences are those identified herein, or a conservatively substituted amino acid sequence of SEQ ID NOs: 59 and/or 60; SEQ ID NOs: 61 and/or 62; or SEQ ID NOs: 63 and/or 64; wherein the CDR sequences are those identified herein. In a further embodiment, the antibody comprises a heavy chain and a light chain having an amino acid sequence with at least 80%, at least 90%, or at least 95% sequence identity to SEQ ID NO: 65 and/or 66, SEQ ID NO: 67 and/or 68, or SEQ ID NO: 69 and/or 70 wherein the CDR sequences are those shown SEQ ID NOs: 71-88; or a conservatively substituted amino acid sequence of SEQ ID NO: 65 and/or 66, SEQ ID NO: 67 and/or 68, or SEQ ID NO: 69 and/or 70, wherein the CDR sequences are those shown in SEQ ID NOs: 71-76, 77-82 and/or 83-88 respectively.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences are maximally aligned by Kabat, IMGT, or other numbering conventions. The terms “Kabat numbering”, “IMGT numbering”, etc., which are recognized in the art, refer to systems of numbering amino acid residues which are more variable (i.e. hypervariable) than other amino acid residues in the heavy and light chain variable regions of an antibody, or antigen binding portion thereof. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Accordingly, Kabat, IMGT, and other alignment systems can also be used to identify or annotate CDRs in an antibody sequence.

A “conservative amino acid substitution” as used herein, is one in which one amino acid residue is replaced with another amino acid residue without abolishing the protein's desired properties. Suitable conservative amino acid substitutions can be made by substituting amino acids with similar characteristics such as hydrophobicity, polarity, and R-chain length for one another.

An aspect of the disclosure includes an isolated nucleic acid molecule encoding an antibody described herein. The nucleic acid molecule may be comprised in a vector. The nucleic acid molecule may for example be incorporated into an expression cassette or expression vector for expression of the antibody. Accordingly, an aspect includes an expression cassette or a vector, optionally an expression vector, comprising a nucleic acid molecule encoding an antibody described herein. Suitable expression vectors include but are not limited to cosmids, plasmids (e.g. pHP153 (Persson et al)), or modified viruses (e.g. replication defective retroviruses, adenoviruses and adeno-associated viruses).

As described in the Examples, IF1 was identified from a naïve library derived from germline framework VH3-30 (variable heavy chain) and VK1 (variable light chain) genes. Other germline sequences can also be used.

As also described in the Examples, IF2 and IF3 are derived from trastuzumab framework which shares similarity to VH3-23 and VK1. Other framework regions can also be used.

The term “nucleic acid” or “nucleic acid molecule”, as used herein, are intended to include unmodified DNA or RNA or modified DNA or RNA. The nucleic acid molecules of the disclosure may contain one or more modified bases or DNA or RNA backbones modified for stability or for other reasons. Unless otherwise indicated, standard IUPAC-IUB nomenclature is used herein. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning. The nucleic acid can be either double stranded or single stranded, and represents the sense or antisense strand. Further, the term “nucleic acid molecule” includes the complementary nucleic acid sequences as well as codon optimized or synonymous codon equivalents. The term “isolated nucleic acid molecules” as used herein refers to a nucleic acid substantially free of cellular material or culture medium when produced by recombinant DNA techniques, or chemical precursors, or other chemicals when chemically synthesized.

In one embodiment, the present disclosure includes functional variants to the nucleic acid molecules disclosed herein. The functional variants include nucleotide molecules that hybridize to the nucleic acid molecules set out herein, under at least moderately stringent hybridization conditions, optionally stringent hybridization conditions.

With reference to nucleic acids, the terms “anneal” and “hybridize” as used herein refer to the ability of a nucleic acid to non-covalently interact with another nucleic acid through base-pairing. The terms “complementary” or “complementary nucleic acid” refer to a nucleic acid or a portion of a nucleic acid that is able to anneal with a nucleic acid of a given sequence. In some cases this is referred to as the “reverse complement” of a given sequence.

By “stringent hybridization conditions” it is meant that conditions are selected which promote selective hybridization between two complementary nucleic acid molecules in solution. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log10[Na+])+0.10(%(G+C)−600/l) or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. By way of example the following conditions may be employed to achieve stringent hybridization: hybridization at 5×sodium chloride/sodium citrate (SSC)/5×Denhardt's solution/1.0% SDS at Tm −5° C. based on the above equation, followed by a wash of 0.2×SSC/0.1% SDS at 60° C. Stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

The term “vector” as used herein comprises any intermediary vehicle for a nucleic acid molecule which enables said nucleic acid molecule, for example, to be introduced into prokaryotic and/or eukaryotic cells and/or integrated into a genome, and include plasmids, phagemids, bacteriophages or viral vectors such as retroviral based vectors, Adeno Associated viral vectors and the like. The term “plasmid” as used herein generally refers to a construct of extrachromosomal genetic material, usually a circular DNA duplex, which can replicate independently of chromosomal DNA.

Also provided herein is a recombinant cell comprising the nucleic acid molecule, expression cassette or vector, optionally an expression vector described herein, the nucleic acid encoding an antibody described herein. The recombinant cell can be prepared by introducing the nucleic acid molecule, expression cassette, vector, optionally expression vector, into a suitable host cell. Preferably, the host cell is suitable for antibody expression or for producing large quantities of the expression cassette or the vector. As would be known to the skilled artisan, a vector compatible with the particular host cell is used.

The recombinant cell can be generated using any host cell suitable for producing a polypeptide, for example suitable for producing an antibody and/or binding fragment thereof. For example to introduce a nucleic acid and/or a vector into a cell, the host cell may be transfected, transformed or infected, depending upon the vector employed. Suitable host cells include a wide variety of prokaryotic and eukaryotic host cells. For example, the antibodies described herein may be expressed in bacterial cells such as E. coli, insect cells (using baculovirus), yeast cells or mammalian cells.

The antibodies described herein may be provided as immunoconjugates. Accordingly, also provided herein are immunoconjugates comprising an antibody described herein and a suitable reagent such as a therapeutic agent, or detectable label. Suitable reagents can be identified by the skilled person depending on the application. In the context of radioimmunotherapy (RIT), the therapeutic agent is a radionuclide, for example an alpha- or beta-emitting radionuclide. Other therapeutic agents are contemplated for use for example as chemotherapeutic agents. Radionuclides may also be suitable labels in the context of in vivo imaging or diagnostic imaging, including for example positron emission tomography (PET), scintigraphic imaging or SPECT, and other imaging techniques. The choice of the particular radioisotope with which the antibody is labeled will depend on the size of the tumor to be treated and its localization in the body. Two characteristics are important in the choice of a radioisotope - emission range in the tissue and half-life. Alpha emitters, which have a short emission range in comparison to beta emitters, may be preferable for treatment of small tumors or tumors that are disseminated in the body. Examples of alpha emitters include 213-Bismuth (half-life 46 minutes), 223-Radium (half-life 11.3 days), 224-Radium (half-life 3.7 days), 225-Radium (half-life 14.8 days), 225-Actinium (half-life 9.9 days), 212-Lead (half-life 10.6 hours), 212-Bismuth (half-life 60 minutes), 211-Astatin (half-life 7.2 hours), and 255-Fermium (half-life 20 hours).

Beta emitters, with their longer emission range, may be preferable for the treatment of a large tumor(s) e.g. greater than 2 mm in diameter. Examples of beta emitters include 188-Rhenium (half-life 16.7 hours), 90-Yttrium (half-life 2.7 days), 32-Phosphorous (half-life 14.3 days), 47-Scandium (half-life 3.4 days), 67-Copper (half-life 62 hours), 64-Copper (half-life 13 hours), 77-Arsenic (half-life 38.8 hours), 89-Strontium (half-life 51 days), 105-Rhodium (half-life 35 hours), 109-Palladium (half-life 13 hours), 111-Silver (half-life 7.5 days), 131-Iodine (half-life 8 days), 177-Lutetium (half-life 6.7 days), 153-Samarium (half-life 46.7 hours), 159-Gadolinium (half-life 18.6 hours), 186-Rhenium (half-life 3.7 days), 166-Holmium (half-life 26.8 hours), 166-Dysprosium (half-life 81.6 hours), 140-Lantanum (half-life 40.3 hours), 194-Irridium (half-life 19 hours), 198-Gold (half-life 2.7 days), and 199-Gold (half-life 3.1 days). The majority of the beta-emitting radioisotopes that are used for radioimmunotherapy can also be used simultaneously for radioimmunoimaging with conventional nuclear medicine equipment such as scintigraphic imaging or SPECT. For example, 177Lu can be used for imaging with SPECT.

A antibody can be for example double-labeled with a diagnostic and a therapeutic radionuclide. Accordingly, also contemplated herein are antibodies which are double-labeled for example with a diagnostic radionuclide and therapeutic radionuclide. For example, an alpha particle emitter such as 225Ac can be used in combination with for example 111In for SPECT imaging, or an alpha particle emitter such as 212Pb/212Bi could be used in combination with 203Pb, also for SPECT imaging.

Positron emitters could be used for radioimmunoimaging techniques such as positron emission tomography (PET). Suitable positron emitters include the following radioisotopes (half-life is indicated in parenthesis): 52mMn (21.1 min); 62Cu (9.74 min); 68Ga (68.1 min); 82Rb (1.27 min); 110In (1 .15 h); 118Sb (3.5 min); 122I (3.63 min); 18F (1.83 h); 34mCl (32.2 min); 38K (7.64 min); 51Mn (46.2 min); 52Mn (5.59 days); 52Fe (8.28 h); 55Co (17.5 h); 61Cu (3.41 h); 64Cu (12.7 h); 72As (1.08 days); 75Br (1.62 h); 76Br (16.2 h); 82Rb (6.47 h); 83Sr (1.35 days); 86Y (14.7 h); 89Zr (3.27 days); 94mTc (52.0 min); 120I (1.35 h); 124I (4.18 days).

Gamma emitters could be also used for radioimmuoimaging techniques such as scintigraphic imaging or SPECT. Suitable gamma emitters include: 99mTc, 111In, 88Y, 67Ga, and 123I.

Other detectable labels, such as fluorescent dyes, enzymes, or biotin may be used depending on the application, and are contemplated herein.

Immunoconjugates may be generated using any suitable technique. Common conjugation techniques include N-hydroxysuccinimide ester (NHS ester) or maleimide crosslinking, but other techniques are known in the art. In the case of radionuclide-conjugated antibodies, bifunctional chelating agents may be used. Suitable bifunctional chelating agents are known in the art, including (R)-2-Amino-3-(4-isothiocyanatophenyl)propyl]-trans-(S,S)-cyclohexane-1,2-diamine-pentaacetic acid (CHXA″). Other suitable chelating agents include DTPA (diethylenetriamine pentaacetic acid), TCMC (1,4,7,10-tetraaza-1,4,7,10-tetra(2-carbamoylmethyl)cyclododecane), TETA (1,4,8,11-Tetraazacyclotetradecane-1,4,8,11-tetraacetic acid), DOTA (1,4,7,10-tetraazacyclododecane tetraacetic acid), macropa (N,N′-bis[(6-carboxy-2-pyridil)methyl]-4,13-diaza-18-crown-6). Optionally, the immunoconjugate is generated using an initial molar ratio, of CHXA″ to antibody of about 2.5 or less, and greater than about 0.3. Suitable molar ratios of other chelators to antibody may range from 0.3 to 100 and include any 0.1 increment there between e.g., 0.4 to 100, 0.4 to 99.9 etc. Different ratios may be used for example, depending on the specific chelator and antibody combination. Initial molar ratio for example for a bifunctional chelating agent such as CHXA″, may be several times more moles of CHXA″ to the moles of the antibody for conjugation. For example, an initial molar ratio of 2.5 means that for 1 nanomole of IF1 or IF3 antibody 2.5 nanomoles of CHXA″ are used in a conjugation reaction.

As will be understood by the skilled person, the molar ratio or chelator-antibody ratio (CAR) refers to the ratio of chelator to antibody in the conjugate. The CAR for examplemay depend on various factors such as the antibody (e.g., the number of lysines present) and the linker used, as well as the initial molar ratio of each used in the conjugation reaction. As described herein for example, for IF3, an initial molar ratio of 2.5 achieved a CAR of 0.91, as shown in Example 3.

Without wishing to be bound by theory, higher levels of CHXA″ in the antibody conjugate may speed up the blood clearance as more CHXA″ molecules on the Fc portion of the antibody would result in less binding to the FcRn receptors on circulating blood cells such as monocytes, and thus will clear faster from the blood. The optimum level of CHXA″ or other chelator conjugated to antibody, (i.e. CAR) would be one which still enables quantitative radiolabeling but does not increase blood clearance. Any suitable CAR may be used, and may depend on the antibody and the particular bifunctional linker. Optionally, a suitable CAR is about 0.7 to about 1.3 or any number or range there between, for example about 0.8 to about 1.2, about 0.9, about 0.91, or about 1. The CAR may be determined for example using mass spectrometry (e.g. MALDI-TOF) or other suitable technique. Achieving the optimum CAR preserves the immunoreactivity of the antibody (e.g. reactivity of IF3 towards IGF2R), permits radiolabeling of the antibody with high specific activity and high yield, while avoiding the need for further purification. This may be important especially from a manufacturing standpoint, as post-labeling purification can result in losses of product on the purification column, dilution of the antibody, etc.

The antibodies and immunoconjugates described herein are shown to be useful for detecting IGF2R in samples, for staining osteosarcoma cells and canine osteosarcoma tumors, and for radionuclide-based therapeutics and in vivo imaging. Accordingly, a further aspect includes the use of the antibodies and immunoconjugates described herein for staining biological samples, imaging, and therapeutics. The skilled person can select an antibody or immunoconjugate with suitable properties depending on the application. For example, an antibody which demonstrates slower clearance from the blood, such as IF3 (SEQ ID NOs: 69 and 70), may be preferred for in vivo applications such as radioimmunoimaging and RIT.

A further aspect includes a library comprising nucleic acid molecules encoding antibodies based on for example the 4D5-8 framework, the nucleic acid molecules defined according to a mutation scheme defined in the Examples.

As described in the examples, mutations were limited to solvent exposed residues, which without being limited to theory may provide a better fraction of the library will be folded compared to other libraries described based for example on the 4D5-8 framework. In an embodiment, the codon used to make the library are (N1)HT. Other codons can also be used, for example to provide alternative amino acid residues and/or frequencies.

### III. Compositions

A further aspect is a composition comprising an antibody, nucleic acid, vector immunoconjugate or library described herein.

In an embodiment the composition comprises a diluent. Suitable diluents for nucleic acids include but are not limited to water, saline solutions and ethanol. Suitable diluents for polypeptides, including antibodies or fragments thereof and/or cells include but are not limited to saline solutions, pH buffered solutions and glycerol solutions or other solutions suitable for freezing polypeptides and/or cells.

In an embodiment, the composition is a pharmaceutical composition comprising any of the antibodies, nucleic acids, vectors, or immunoconjugates disclosed herein, and optionally comprising a pharmaceutically acceptable carrier. Pharmaceutically acceptable carriers include for example include any and all solvents, dispersion media, isotonic and absorption delaying agents, and the like that are physiologically compatible. a The pharmaceutically acceptable carrier can be water or a buffered saline, with or without a preservative.

The composition may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The term “pharmaceutically acceptable” means compatible with the treatment of animals, in particular, humans.

The compositions described herein can be prepared by per se known methods for the preparation of pharmaceutically acceptable compositions that can be administered to subjects such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

### IV. Kits

Also provided are kits comprising the antibody, nucleic acid, vector, cell, immunoconjugate, library or composition as described herein, along with suitable container or packaging and/or instructions for the use thereof, such as for the detection or treatment of cancer in a subject.

In an embodiment, the kit comprises reagents and/or instructions for use in a method of IGF2R detection such as ELISA or IHC.

### V. Methods

The antibodies described herein are shown herein for example to be cross-reactive for human, murine, and canine IGF2R, to bind human- and canine-derived osteosarcoma cell lines, and to stain canine osteosarcoma tumors. Accordingly, an aspect includes a method of detecting IGF2R expression in a biological sample, the method comprising obtaining a biological sample, contacting the sample with an antibody or immunoconjugate described herein under conditions permissive for forming an antibody:IGF2R complex, and detecting the presence of an antibody complex. A further aspect includes a method of detecting whether a subject has an IGF2R-expressing cancer, the method comprising obtaining a biological sample suspected of containing an IGF2R-expressing cancer cell from the subject, contacting the sample with an antibody or immunoconjugate described herein under conditions permissive for forming an antibody:IGF2R complex, and detecting the presence of an antibody complex, wherein the presence of an antibody complex indicates that the subject has an IGF2R-expressing cancer. In an embodiment, the IGF2R-expressing cancer is selected from osteosarcoma, pleomorphic adenoma, intestinal adenoma, medulloblastoma, adenocortical carcinoma, mucosal melanoma, and hepatocellular carcinoma. In an embodiment, the IGF2R-expressing cancer is osteosarcoma.

Suitable biological samples include, without limitation a tissue sample such as a tumor sample, which can be a solid tissue biopsy such as a liver biopsy, a kidney biopsy, a bone marrow biopsy, and a bone biopsy, or a liquid biopsy such as a blood sample or plasma sample. A blood sample or plasma sample may be used for example for detecting circulating tumour cells or circulating exosomes. In an embodiment, the biological sample is a tumor sample. Suitable methods for obtaining tissue samples include tissue biopsy, fine needle aspiration cytology, fluid cytology, needle biopsy, CT-guided biopsy, ultrasound-guided biopsy, aspiration biopsy, liver biopsy, kidney biopsy, bone marrow biopsy, or bone biopsy. Testing for IGF2R expression can be done by any suitable analytic technique, including immunohistochemistry or flow cytometry.

In an embodiment, the sample is obtained from a human or canine subject, optionally the subject has or is suspected of having a cancer such as osteosarcoma, pleomorphic adenoma, intestinal adenoma, medulloblastoma, adenocortical carcinoma, mucosal melanoma, or hepatocellular carcinoma.

Also shown herein, radio-labeled antibodies described herein are useful for microSPECT/CT imaging of human osteosarcoma tumors in mice. Accordingly, an aspect includes a method of imaging an IGF2R-expressing tumor, optionally the cancer is selected from osteosarcoma, pleomorphic adenoma, intestinal adenoma, medulloblastoma, adenocortical carcinoma, mucosal melanoma, and hepatocellular carcinoma, preferably the cancer is osteosarcoma, in a subject, the method comprising administering an immunoconjugate or composition comprising an immunoconjugate described herein to the subject, and detecting the presence of the label by imaging. In an embodiment, the label is a radionuclide, optionally 111Indium. A further aspect includes a method of determining if a subject has an IGF2R-expressing tumor, the method comprising administering an immunoconjugate or composition comprising an immunoconjugate described herein to the subject, and detecting the presence of the label by imaging. In an embodiment, the subject has or is suspected of having an IGF2R-expressing cancer, optionally osteosarcoma.

Any suitable imaging technique may be used and will depend on the label. For example, SPECT/CT can be used to image immunoconjugates labeled with radionuclides such as 111Indium. Other suitable imaging techniques include for example SPECT, PET, PET/CT, PET/MRI, scintigraphy, and planar imaging.

In an embodiment, the detection method is used to monitor disease state, burden, progression, or remission in a subject.

Immunoconjugates comprising the antibodies and a radionuclide are shown herein to be cytotoxic to osteosarcoma cells. The antibodies described herein may also be useful for treating IGF2R-expressing cancers such as osteosarcoma. Accordingly, a further aspect is a method of treating a cancer, optionally the cancer is selected from osteosarcoma, pleomorphic adenoma, intestinal adenoma, medulloblastoma, adenocortical carcinoma, mucosal melanoma, and hepatocellular carcinoma, preferably the cancer is osteosarcoma, the method comprising administering an effective amount of an antibody, immunoconjugate, or composition described herein to a subject in need thereof. In an embodiment, the immunoconjugate comprises an antibody comprising the light and heavy chains of SEQ ID NOs: 69 and 70 and a radionuclide, optionally 111Indium or 177Lutetium.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. In one embodiment, treatment methods comprise administering to a subject a therapeutically effective amount of an antibody or immunoconjugate described herein, and optionally consists of a single administration, or alternatively comprises a series of administrations.

As used herein, the term “cancer” refers to one of a group of diseases caused by the uncontrolled, abnormal growth of cells that can spread to adjoining tissues or other parts of the body. Cancer cells can form a solid tumor, in which the cancer cells are massed together, or exist as dispersed cells. Cancer may include osteosarcoma, pleomorphic adenoma, intestinal adenoma, medulloblastoma, adenocortical carcinoma, mucosal melanoma, and hepatocellular carcinoma, among others. In some embodiments the cancer is osteosarcoma.

The term “cancer cell” refers to a cell characterized by uncontrolled, abnormal growth and the ability to invade another tissue or a cell derived from such a cell. Cancer cells include, for example, a primary cancer cell obtained from a patient with cancer or cell line derived from such a cell. In one embodiment, the cancer cell is an osteosarcoma cell.

The term “administered” as used herein means administration of a therapeutically effective dose of an antibody, immunoconjugate, or composition of the disclosure to a cell or subject. The antibodies, immunoconjugates, compositions, etc. described herein can be administered for example, by parenteral, intravenous, subcutaneous, intramuscular, intracranial, intraventricular, intrathecal, intraorbital, ophthalmic, intraspinal, intracisternal, intraperitoneal, intranasal, aerosol or oral administration. In certain embodiments, the pharmaceutical composition is administered systemically.

As used herein, the phrase “effective amount” or “therapeutically effective amount” means an amount effective, at dosages and for periods of time necessary to achieve the desired result. For example in the context of treating cancer such as osteosarcoma, an effective amount is an amount that for example induces remission, reduces tumor burden, and/or prevents tumor spread or growth of cancer cells compared to the response obtained without administration of the compound. Effective amounts may vary according to factors such as the disease state, age, sex and weight of the subject. The amount of a given compound that will correspond to such an amount will vary depending upon various factors, such as the given antibody or immunoconjugate, the pharmaceutical formulation, the route of administration, the type of disease or disorder, the identity of the subject or host being treated, and the like, but can nevertheless be routinely determined by one skilled in the art.

The term “subject” as used herein includes all members of the animal kingdom including mammals, and suitably refers to humans and canines. Optionally, the term “subject” includes mammals that have been diagnosed with cancer, such as osteosarcoma, or are in remission.

In one embodiment, the term “subject” refers to a human having, or suspected of having, cancer such as osteosarcoma. In another embodiment, the term “subject” refers to a canine having, or suspected of having, cancer such as osteosarcoma.

In an embodiment the subject being treated is tested for the presence of IGF2R-expressing cells or tumors using the detection and/or imaging methods described herein. For example, imaging or image based dosimetry can be used. In an embodiment, the subject is tested before, during, or after treatment. Optionally, the subject is tested at multiple time points.

Solid tumors such as OS have been reported to require high doses of beta-emitters such as 177Lu to achieve a therapeutic effect. For example, Lutathera™, which is a clinically approved peptide labeled with 177Lu for treatment of neuroendocrine tumors, is administered as 200 mCi per administration, and is given 4 times with 8 weeks between administrations.

In an embodiment, the subject is treated with low dose 177Lu immunoconjugate, for example less than about 40 mCi, 30, mCi, or 20 mCi per administration for example less than or about 15 mCi or 14 mCi per administration in a 70 kg adult human or less than or about 7.5 mCi or 7 mCi per administration in a 35 kg pediatric child. The 177Lu immunoconjugate may be administered for example 1-4 times with about 4-8 weeks between administrations.

In an embodiment, a dosimetry calculation is performed to determine the radiation dose, extrapolated and calculated from for example mouse data, using suitable methods. For example, the average percent administered activity per gram in the mouse can be used to calculate an extrapolated set of values as described in Molina-Trinidad et al. [21] and Example 3.

In an embodiment, the subject is pretreated with unlabeled or non-radiolabeled antibody (i.e. “cold” antibody) before administration of RIT. Such “preblocking” with cold antibody can be done in the clinic before administration of RIT for example to block non-cancerous sites which may express target antigen.

Also provided are uses of the antibodies, immunoconjugates, and compositions for detecting and/or treating cancer such as osteosarcoma. Also provided are use of immunoconjugates described herein for radioimmunotherapy (RIT).

The following non-limiting examples are illustrative of the present application:

### VI. EXAMPLES

### Example 1

### Generation of Cross-Reactive Antibodies to Human, Murine and Canine IGF2R.

Construction of synthetic and naïve antibody Fab phage-display libraries: A custom phagemid based on previously published pHP153 (Persson et al) was used for cloning and display of antibody variants in Fab format. This phagemid is partly derived from pBR322 and drives expression of antibody variants as a fusion to truncated gene 3 protein (C-terminal domain) under phoA promoter.

For the synthetic library construction, humanized Her2 clone 4D5-8 (Trastuzumab) (Carter et al.) framework cloned in pHP153 was used as a template for mutagenesis. The design of synthetic library is shown in FIG. 1. Based on trastuzumab crystal structure (pdb 1N8Z), solvent exposed positions in complementary determining regions (CDRs) were selected for mutagenesis. CDRL3 length was allowed to vary between 9-12 amino acids and CDRH3 was allowed to vary between 12-19 amino acids as described in FIG. 1. The 4D5-8 synthetic library was constructed by site-directed mutagenesis using mutagenic oligonucleotides ordered from Integrated DNA Technologies (IDT). All randomized positions were mutated using oligonucleotides with a custom (N1)HT codon, where N1 is a mix of 10% A, 20% C, 25% G and 45% T. The DNA and amino acid sequences of the 4D5-8 Fab template (VH and VL regions) are provided in SEQ ID NOs: 1-4, and mutagenic oligonucleotides used for library construction are provided in SEQ ID NOs: 5-20. The library was constructed using previously described methods using site-directed mutagenesis (Persson et al; Fellouse et al), except that all six CDRs were mutated in the same reaction. Oligonucleotides for CDRL3 and CDRH3 were pooled together as they anneal to same region. Given the high number of oligonucleotides for CDRH3 randomization, the reaction was split into two mixes (Set1: 4D5-Lib1-H3-4aa to 4D5-Lib1-H3-7aa; Set2: 4D5). Two 10 μg scale reactions were conducted with each set of CDRH3 mixes. The covalently coupled double-stranded DNA from both reactions was then pooled together and electroporated into E.Coli. SS320 that were pre-infected with helper phage. Approximately 3.3×10⁹ transformants were obtained.

For naïve library construction, a set of primary PCR primers were used to amplify genes encoding antibody variable regions using cDNA derived from pooled peripheral blood monocytes of human blood donors (purchased from Takara Bio) according to previously described protocols (Hust et al). Briefly, the antibody repertoire from pooled human peripheral leukocyte poly A+RNA (Takara Bio) was cloned using Thermoscientific Maxima H Minus First strand cDNA synthesis kit to generate single-strand cDNA template from 500 ng poly A+RNA using kit instructions. The primers provided in SEQ ID NOs: 21-39 were used to generate primary PCR products corresponding to VH and VL repertoire.

200 μl scale PCR reactions were set up using Phusion DNA polymerase using manufacturer recommended protocols for each of VK1, VK3 and VH3. Forward and Reverse primers for each subfamily as listed in the table were pooled together. 2.5 μl of first-strand cDNA was used as a template and a gradient annealing temperature of 63-72° C. was used for each PCR reaction. Following electrophoresis, bands of ˜680 bp for VL and ˜380 bp for VH were excised from the gel and purified using gel extraction kit.

A set of secondary PCR primers with vector specific overhangs were used to amplify VH and VL regions which were then spliced with intergenic constant region (containing CH1 and IRES sequences) using splice-overlap-extension PCR. Briefly, a secondary PCR with overhangs to add restriction sites was setup at the 400 μl scale with 200 ng of primary PCR product as the template using primers provided in SEQ ID NOs: 40-57. Following secondary PCR, bands of ˜380 bp for VK1A/K3 and ˜400 bp for VH3 were gel-extracted and purified. 200 ng of light chain and heavy chain PCR products were spliced with 300 ng of dsDNA with intergenic sequence provided in SEQ ID NO: 58 using Splicing Overlap Extension (SOE) PCR.

Following SOE PCR with light-chain forward primer pool and heavy chain reverse primer pool, a band corresponding to the assembled product (1250 bp) was gel extracted and purified. Vector pHP153 and purified PCR products were digested with restriction enzymes Nsil and Nhel and purified using gel-extraction kit. Two ligation reactions (K1 and K3) were setup using 1.5 μg vector and 2.5 μg insert to give a molar ratio of (1:5) using T4 DNA Ligase at 16° C. overnight. The DNA was purified on a QIAquick columns and pooled together for electroporation. The ligated DNA was then electroporated into bacteria for phage library construction as described previously (Fellouse et al). Following electroporation ˜4×10⁸ transformants were obtained. Sequencing analysis showed that only 25% of clones had inserts giving a library size of 10⁸ variants.

Production of recombinant human, murine and canine IGF2R fragments: Gene fragments encoding the domains 11-13 of human IGF2R (SEQ ID NO: 89, corresponding to aa 1511-1989, Uniprot P11717), murine IGF2R (SEQ ID NO: 90, corresponding to aa 1504-1982, Uniprot Q07113) and canine IGF2R (SEQ ID NO: 91, corresponding to aa 1515-1993, Uniprot B1H0W0) were generated using gene synthesis. These sequences were cloned into a modified pFUSE-hIgG1-Fc2 vector (Invivogen) for generation of soluble Fc fusion proteins. Recombinant proteins were expressed in Expi293F cells (Invitrogen) and purified using MabSelectSure resin (GE Healthcare), using manufacturer recommended protocols.

Phage library panning and ELISA. A modified approach was used to select for antibody variants that recognize IGF2R fragments from different species. The selection and ELISA methodology is essentially the same as described previously (Fellouse et al), with the modification that antigens were swapped every round. Human IGF2R fragment was used for Round 1 of selection, murine for Round 2 and canine for Round 3. Following 3 rounds of selection, 48 clones were analyzed from naïve library selection pool. For synthetic library, an additional round of selection was performed on human IGF2R fragment. 48 clones from synthetic library selection Round 4 pool were analyzed using ELISA.

ELISA with recombinant IGF2R. The coating concentration for the recombinant human, murine and canine IGF2R was 3 ug/ml with 50 ul (0.15 ug) added to each well. The plate was blocked with 100 ul PB Buffer (1×PBS+0.2 mg/ml BSA) and left for one hour at room temperature. The buffer was discarded and washed the plate with PT buffer (1×PBS+Tween) twice then add primary antibody. 50 ul of the primary antibody was added to the corresponding wells and incubated for 1 hour at room temperature while shaking. The plate was washed 4 times with PT Buffer using plate washer. The Goat Anti-Human Kappa (K) Light Chain Antibody HRP secondary antibody was diluted (1:5000) in PBT buffer (1×PBS, 0.2 mg/ml BSA+Tween)). 50 ul secondary antibody was added to each well. The plate was incubated for 45 mins at room temperature while shaking. The plate was washed with PT four times using plate washer. 45 ul TMB (1:1) was added to each well for ˜5 mins. 45 ul 1 M Phosphoric acid was added to each well to terminate reaction and the plate was read.

Production of Fab fragments in bacteria. The coding sequences for the VL and VH regions of selected Fabs (SEQ ID NOs: 59-64) were cloned into a custom expression vector under with protein expression driven with a ptac promoter, with a C-terminal 6xHis tag at the end of CH1 domain on heavy chain. Fab proteins were expressed in E.Coli. BL21 Codon Plus cells (Agilent). Actively growing cells (O.D. 0.8) were induced using 0.4 mM IPTG and grown at 24° C. for 12 h. Cells were lysed and the fab proteins were purified from clarified lysate using Ni-NTA Sepharose resin (GE Healthcare) using manufacturer recommended protocols.

Radiolabeling of Fabs. Fabs were conjugated with CHXA″ and radiolabeled as described below in Example 2 and Example 3 for the full-length antibodies.

### Results

### Generation of Cross-Reactive Antibodies to Human, Murine and Canine IGF2R.

Following phage library panning, 1 sequence (IGF2R-Fab-1) was isolated from the naïve library that repeated multiple times. Several hits were obtained from synthetic libraries, but two clones (IGF2R-Fab-2, IGF2R-Fab-3) that showed comparable ELISA signals to all IGF2R fragments were selected. Phage ELISA confirmed specific binding of isolated Fab-phage to IGF2R with no binding to control Fc fusion proteins (FIG. 3A). Competitive Fab-phage ELISA revealed tight binding to human, murine and canine IGF2R recombinant proteins (FIG. 3B). The annotated sequences of the isolated Fabs are provided in SEQ ID NOs: 59-64.

Fab′ Fragments Bound to IGF2R and were Cytocidal to IGF2R Positive Cells in Vitro.

Purified Fab proteins, when radiolabeled with the alpha-emitter 225Ac-Fab1 killed IGF2R-positive 143B human OS cells in a dose dependent manner (FIG. 4A) while no killing of IGF2R-negative K7M2 murine OS cells was observed (FIG. 4B).

### Example 2

Production of full-length IqG in human cells. For production of IgG version of lead antibodies, the VL and VH coding sequences were cloned into pFUSE2ss-CLIg-hK vector and pFUSE2ss-CHIg-hG1 vectors respectively (Invivogen). The sequences of the IgG clones are provided in SEQ ID NOs: 65-70. These clones were expressed in Expi293F cells using previously described protocols (Vasquez-Lombardi et al). Purity of the proteins was analyzed on SDS-PAGE gels.

Reagents and antibodies. 2G11 mAb was obtained from ThermoFisher (Canada). Human mAb Palivizumab (IgG1) against respiratory syncytial virus (RSV) was acquired from MedImmune and was used as an isotype-matching negative control. (R)-2-Amino-3-(4-isothiocyanatophenyl)propyl]-trans-(S,S)-cyclohexane-1,2-diamine-pentaacetic acid (CHXA″) BCA was purchased from Macrocyclics (USA). ¹¹¹In was obtained from Nordion (Canada); and ²²⁵AC-from Oak Ridge National Laboratory (USA). Silica gel instant thin layer chromatography (SG-iTLC) strips were obtained from Agilent (Canada).

Cell lines. Human and canine osteosarcoma cell lines 143B and D17, respectively, were obtained from American Type Culture Collection (ATCC, Manassas, VA, USA). OS-33, a well characterized patient-derived osteosarcoma cells line, was obtained from Dr. R. Gorlick (MD Anderson Cancer Center). McKinley and Gracie canine osteosarcoma cell lines were obtained from Dr. Behzad Toosi, U of S. Cells were cultured in Eagle's Minimum Essential medium and supplemented with 10% FBS, sodium pyruvate, non-essential amino acids, and 100 U penicillin/0.1 mg/ml streptomycin.

Animal models. All animal studies were approved by the Animal Research Ethics Board of the University of Saskatchewan (#2017006). All animal experiments were performed in accordance with the Canadian Council on Animal Care guidelines for humane animal use. Healthy six-eight week old SCID (CB17/Icr-Prkdc^{scid}/IcrIcoCrI) female mice obtained from Charles River Laboratories (USA) were used for the biodistribution experiments. For tumor induction the mice were anesthetized with isoflurane and injected subcutaneously with 3×10⁶ 143B cells into the right flank. Mice were monitored for tumor development, and it was noted that for the 143B cell line 80% of mice developed palpable tumors by day 12.

Conjugation of bifunctional chelating agent CHXA″ to mAbs. 10X conjugation buffer (0.05 M Carbonate/Bicarbonate, 0.15 M NaCl, 5 mM EDTA, pH 8.6-8.7), 5 mL was combined with 0.5 M EDTA, pH=8.0 (0.5 mL) and was diluted to 50 mL in a 50 mL Falcon tube with deionized water to give the 1X buffer. An Amicon Ultra 0.5 mL centrifugal filter (30K MW cut off, Fisher) was loaded with 2 mg of either 2G11 or MOPC-21 antibody. The antibody was exchanged into the above conjugation buffer by performing 6×1.5 mL washes using an Amicon concentrator in a refrigerated centrifuge at 4° C. A solution of bifunctional CHXA″ ligand with 2 mg/mL concentration was prepared by dissolving CHXA″ in conjugation buffer. The antibody was recovered from the Amicon and 23.6 pL of 2 mg/mL CHXA″ solution in conjugation buffer is added to provide 2.5 or 10 fold molar excess of CHXA″ over the antibody. The reaction mixture was incubated at 37° C. for 1.5 hrs. The reaction mixtures was then purified into 0.15 M ammonium acetate buffer, pH=6.5-7.0, with 6×1.5 mL washes on Amicon concentrators in a refrigerated centrifuge at 4° C. The sample were stored at 4° C. A Bradford assay was performed to determine protein recovery and concentration.

Radiolabeling of mAbs. The mAbs were radiolabeled with 111In as described in (Karkare et al), and original Fabs were radiolabeled with 225Ac for in vitro cell killing as in (Garg et al). The percentage of radiolabeling was measured by SG-iTLC using 0.15 M ammonium acetate buffer as the eluent. SG-iTLCs were cut in half and read on a Perkin Elmer 2470 Automatic Gamma Counter (top containing unlabeled ¹¹¹In or 225Ac, bottom containing antibody labeled with ¹¹¹In or 225Ac).

Biodistribution of 111In-labeled IF1 and IF3 in healthy mice. Healthy mice were randomized into groups of 5 animals and intraperitoneal (IP) injected with 20 μCi of either ¹¹¹In-IF1, or ¹¹¹In-1F3 prepared with 2.5 or 10 initial molar ratio of CHXA″ to the protein. At 24 and 72 hr post injection 5 mice were sacrificed from each group. Once sacrificed, the blood, spleen, and femur were collected, weighed, and counted in a gamma counter (Perkin Elmer). The percent of injected dose per gram (%ID/g) for each sample was calculated.

microSPECT/CT imaging of 111In-labeled IF3 in human tumors in mice. microSPECT/CT (micro single photon emission computer tomography/computer tomography) images were collected on a MI Labs VECTor⁴ (Netherlands) microSPECT/CT scanner and processed using the comprehensive image analysis software package PMOD (version 3.9, PMOD Technologies, Inc, Switzerland). Imaging studies were conducted using 200 μCi ¹¹¹In-IF3. 143B tumor-bearing mice were administered ¹¹¹In-1F3 via IP injection and imaged in the prone position at 48 hours post injection. SPECT data was collected for 20 minutes using an Extra Ultra High Sensitivity Mouse (XUHS-M) collimator for 20-350 keV range using spiral trajectories. All SPECT images were reconstructed using both 245 keV and 171 keV ¹¹¹In gamma emissions on a 0.4 mm voxel grid with MILabs reconstruction software.

Immunohistochemistry of OS canine tumors with IF1. Immunohistochemical detection of IGF2R in canine OS tumors was performed using an automated staining platform (Autostainer Plus, Dako Canada Inc., Mississauga, ON). Endogenous peroxidase activity was quenched using 3% hydrogen peroxide in methanol. Heat-induced epitope retrieval was performed in a Tris/EDTA pH 9 buffer for 20 min. The tissue was incubated with 1:25 dilution of IF1 mAb overnight at 4° C. Binding of the primary antibody was detected using goat anti-human immunoglobulins (Vector Labs; Burlingame, CA) and an avidin-biotin immunoperoxidase complex reagent (Vector Labs; Burlingame, CA). The staining was visualized using 3,3′-diaminobenzidine tetrahydrochloride (DAB) as the chromogen (Agilent Technologies Canada Inc., Mississauga, ON). Pavilizumab was used instead of the primary antibody as the isotype negative control.

### Results

Full size human IgGs bound to IGF2R from different species, to human and canine OS cells and patient derived cell lines, and to canine tumors. The full-size human IgGs from Fab1 and Fab3 (referred further in the text as IF1 and IF3) were characterized using SDS-PAGE (FIG. 5A, B) and demonstrated specific binding to human, canine and murine IGF2R by ELISA (FIG. 5C, D). Flow cytometry showed that IF1 and IF3 specifically bound to commercial OS cell lines and patient-derived cell lines from human and canine patients (FIG. 6). FIG. 7 shows IHC of IF1 binding to randomly selected tumors from companion dogs with OS while control human mAb pavilizumab demonstrated no or very weak binding (FIG. 7).

Conjugation of IF1 and IF3 linker CHXA″ did not interfere with their immunoreactivity towards IGF2R. Next IF1 and 1F3 were conjugated with the bifunctional linker CHXA″ which enables radiolabeling of the mAbs with diagnostic and therapeutic radionuclides. Comparative ELISA revealed that both antibodies also preserved significant percentage of their immunoreactivity when conjugated to 2.5 and 10 initial molar ratios of CHXA″ bifunctional linker to the antibody, with 2.5 molar ratio having lesser effect on the immunoreactivity of the mAbs (FIG. 8).

IF1 and IF3 showed different clearance from the blood. The IF1 and IF3 mAbs conjugated with 2.5 and 10 initial molar ratio of CHXA″ to the antibody were radiolabeled with 111In and a pilot biodistribution was performed in healthy female SCID mice at 24 and 72 hrs post mAbs administration to evaluate mAbs uptake and clearance from the blood, spleen, and bone. The major difference between IF1 and IF3 was much slower blood clearance of IF3 in comparison with IF1 —at 24 hrs post injection there was only 0.7% ID/g of IF1 in the blood compared to 4.7% ID/g of IF3. Both antibodies showed considerable uptake in the spleen and some uptake in the bone, which decreased from 24 to 72 hrs. The higher amount of CHXA″ ligand attached to either antibody slightly increased the clearance from the blood (FIG. 9). As slower clearance from the blood is thought to be preferred for imaging and therapy, IF3 conjugated to 2.5 initial CHXA″ to mAb molar ratio was chosen for further experiments.

IF3 localized in 143B human OS tumor. 111In-labeled IF3 was administered to female mice bearing 143B human OS tumors in their right flank. FIG. 10 shows the 48 hrs post inject image with clearly visible tumor uptake (FIG. 10). Significant uptake in the spleen was also observed.

### Example 3

Antibody, radionuclides and radiolabeling. Human, canine and murine IGF2R-binding IF3 mAb was expressed and purified as described in Example 1. SG-iTLC strips (Silica gel instant thin layer chromatography) for quantification of radiolabeling yields were acquired from Agilent (Canada). (R)-2-Amino-3-(4-isothiocyanatophenyl)propyl]-trans-(S,S)-cyclohexane-1,2-diamine-pentaacetic acid (CHXA″) bifunctional chelating agent was purchased from Macrocyclics (USA). 111In was obtained from BWXT (Canada); and 177Lu from Radiomedix (USA). IF3 mAb was conjugated to 2.5 initial molar excess of CHXA″ as in Example 2. The conjugate antibody ratio (CAR) of CHXA″ molecules per IF3 molecule post conjugation was determined by MALDI-TOF (University of Alberta, Edmonton, Canada) to be 0.91. This ratio allows for example immunoreactivity of the conjugated IF3 towards IGF2R, and high radiolabeling yield without for example the need for purification. Radiolabeling with 111In and 177Lu was performed as in Example 2. Radiolabeling yields were typically greater than 98% and radiolabeled mAbs required no further purification.

Animal Models. Healthy six to eight-weeks old SCID (CB17/Icr-Prkdcscid/IcrIcoCrI) female mice obtained from Charles River Laboratories (Wilmington, MA, USA) were used for the biodistribution and therapy experiments. Gracie canine patient derived osteosarcoma cell line was a kind gift from Dr. Doug Thamm's lab at Colorado State University School of Veterinary Medicine, USA. Gracie cells were grown as in Example 2. Mice were anesthetized with isofluorane for tumor induction and were injected with 4×10⁶ of Gracie cells into the right flank of the mice. Mice were monitored thrice a week for tumor development.

Biodistribution and microSPECT/CT of 111In-Labeled IF3 in tumor-bearing mice. Mice bearing Gracie tumor were randomized into groups of five and were injected intraperitoneally with 18 μCi of 111In-IF3. At 2, 24, 48, and 72 h post injection, mice were sacrificed, and the following organs were collected: blood, tumor, heart, lungs, pancreas, spleen, kidney, liver, brain, stomach, small intestine, large intestine, thigh muscle and bone. The percentage injected dose per gram (%ID/g) was then calculated by weighing the organ and counting the radioactivity with a gamma counter (Perkin Elmer, Waltham, MA, USA). microSPECT/CT (micro single photon emission computer tomography/computer tomography) images were collected on a MlLabs VECTor4 (Netherlands) microSPECT/CT at 24 and 48 hrs post 111In-IF3 administration (200 μCi via intravenous injections) and processed as in Example 2.

Dosimetry calculations. Radiation doses for human and canine subjects were extrapolated and calculated from the mouse data. MIRD formalism, implemented using OLINDA v2.0, and other direct principles, were used for dosimetry calculations. The average percent administered activity per gram (%IA/g) in the mouse was obtained and group-averaged for each time point and each organ or tissue. We used the relative-organ-mass scaling method of Molina-Trinidad et al. to calculate an extrapolated set of values, repeated at each time point, for the organ masses given in the 9.45 kg canine and the 32 kg pediatric models, as follows:

[(%A/m(g, organ))human]=[(%A/m(g, organ))mouse×m(kg, body)mouse]×(m(g, organ)/m(kg, body))human

[(%A/m(g, organ))canine]=[(%A/m(g, organ))mouse×m(kg, body)mouse]×(m(g, organ)/m(kg, body))canine

where A is the activity administered, measured at each time point, and m is the organ mass (grams) or whole-body mass (kg). The dosimetry calculations extrapolating from mice to canines and humans can be used for example to predict which organs should be assessed when imaging other subjects for example canine or human patients.

The above scaling formula assumes that the metabolic retention and clearance from the mouse can be extrapolated to a human based on the mass ratios given above. This method has been widely used and cited by others; however, direct extrapolation between species based on ratios of organ mass remains tentative and perhaps not fully representative of potential differences in species' metabolic rates. Using this method, new datapoints for (1) the 9.45 kg model and (2) the 32 kg female pediatric model were obtained. The new datapoints for each phantom model were then fitted by least-squares regression analysis to a preferred function and the function was integrated to infinity. The time-integrated activity coefficients for each organ or tissue were entered into OLINDA [22] for the dog model and for the pediatric human model. Radiation doses for ¹⁷⁷Luwere obtained in units of either mSv/MBq organ dose equivalent or centigray (cGy) absorbed dose per millicurie administered. Differences in calculated results occur because of differences in the phantom models. Model assumptions are variable between the two phantoms for heart, heart wall, small intestine and large intestine (such as right and left colon). Radiation doses for muscle and blood were calculated from first principles using the time-integrated activity coefficients for ¹⁷⁷Lu in each, since neither of the phantom models employs blood or muscle as source-target organ pairs.

Therapy of tumor-bearing mice with 177Lu-Labeled IF3 in. Mice were monitored and randomized into groups of five once tumor size reached ˜50-100 mm³. Therapy study included four groups: Group 1 was untreated; group 2 received unlabeled IF3 (cold); group 3 received 60 μCi of 177Lu -IF3; and group 4 received 60 μCi of 177Lu -IF3 with pre-blocking. Mice in group 4 received 200 μg of the IF3 antibody 2 h before treatment. Tumors and mouse body weights were then monitored three times a week. The formula V=L*WA\hat{ }2/2 was used to calculate the tumor volume for each mouse. Mice were humanely sacrificed when they reached the Humane Intervention Point (HIP), namely, the animals were humanely euthanized if they experienced excessive weight loss (20%), became moribund, or any tumor reached 4000 mm 3 volume or became necrotic.

Immunohistochemistry. The IGF2R-specific mAb 2G11 and the isotype matching control mAb MOPC21 were obtained from ThermoFisher (Canada). Immunohistochemistry of the spleens from two dogs was performed as described in Karkare S et al. with the only difference of using 1:100 2G11 mAb dilution. Canine placenta was used as a positive control for IGF2R by analogy with a human placenta (Harris L K et al.).

Statistical analysis. Power analysis for the in vivo studies was estimated using PASS version 11 (NCSS, Inc.) using simulations of different tumor volumes based on pilot data and conservative assumptions regarding the groups treated with the radiolabeled antibodies. All simulations showed power of at least 83% with only five animals per group because of the large differences between treated and untreated animals. Thus, five mice per group were utilized in the in vivo studies. GraphPad Prism 7 was used to analyze all the data (GraphPad Software, Inc.). Differences between the treated and untreated groups in vitro were assessed using nonparametric Kruskal-Wallis test with Dunn's correction for multiple comparisons. Error bars represent±standard deviation (SD). Kaplan-Meier data were analyzed by log-rank (Mantel-Cox) test.

### Results

Biodistribution and microSPECT/CT imaging demonstrated high uptake of IF3 antibody in canine patient derived Gracie tumors. FIG. 11 shows the results of the biodistribution of 111In-IF3 in female SCID mice bearing canine patient derived Gracie tumor. The highest tumor uptake of approximately 18% ID/g was reached at 24 hrs post injection. At all timepoints, the tumor to blood uptake ratio was >4. The IGF2R expressing spleen uptake also reached its highest value of 50% ID/g at 24 hrs post administration. Accumulation of 111In-IF3 in the pancreas, small intestines, bone, and the spine reached its peak values at 48 hrs with washout observed at 72 hrs. Metabolic organs (kidney, liver, stomach) uptake was unremarkable. The only organ which displayed continuous uptake of the radiolabeled antibody were the lungs. The microSPECT/CT imaging of the tumor bearing mice also demonstrated 111In-IF3 uptake in the tumor and the spleen (FIG. 12).

Dosimetry calculations identified the organs to receive the highest radiation dose in a canine and pediatric patient. Radiation absorbed doses were calculated from the mouse biodistribution data and projected to the dog and child using methods consistent with the recommendations of the special committee on Medical Internal Radiation Dose (MIRD) of the Society of Nuclear Medicine and Medical Imaging (SNMMI). Table 1 displays the radiation doses which would be delivered by 177Lu-IF3 mAb to the OS tumor and major organs, calculated by using the biodistribution data described above as applied to the model of a 32 kg 10-year-old female child and a 9.45 kg dog. The organs which would receive the highest radiation dose in the course of RIT of a 10-year-old female child would be in descending order—spleen, tumor, skeletal surfaces, pancreas and lungs. In an average size dog these organs in the descending order would be—tumor, spleen, pancreas. The projected total body dose would be approximately 2 times higher in a pediatric patient than in a canine patient.

TABLE 1
Radiation doses which would be delivered by 177Lu-
IF3 mAb to the OS tumor and major organs in a 32
kg 10 year old female child and a 9.45 kg dog.

| | 32 kg human model | | 9.45 kg dog model | |
|---|---|---|---|---|
| Target Organ | mSv/MBq | cGy/mCi | mSv/MBq | cGy/mCi |
| Adrenals | 4.34E−02 | 1.61E−01 | — | — |
| Brain | 1.07E−02 | 3.96E−02 | 7.40E−02 | 2.74E−01 |
| Heart Wall | 4.69E−02 | 1.74E−01 | 1.66E−01 | 6.14E−01 |
| Eyes | 1.66E−03 | 6.14E−03 | 2.04E−02 | 7.55E−01 |
| Gallbladder Wall | 3.76E−03 | 1.39E−02 | 4.54E−02 | 1.68E−01 |
| Small Intestine | 6.40E−02 | 2.37E−01 | 7.53E−02 | 2.79E−01 |
| Stomach Wall | 1.16E−02 | 4.29E−02 | 8.48E−02 | 3.14E−01 |
| Large Intestine | — | — | 1.20E−01 | 4.44E−01 |
| Right colon | 6.95E−02 | 2.57E−01 | — | — |
| Rectum | 1.16E−03 | 4.29E−03 | — | — |
| Kidneys | 8.86E−02 | 3.28E−01 | 8.85E−02 | 3.27E−01 |
| Liver | 6.48E−02 | 2.40E−01 | 6.38E−02 | 2.36E−01 |
| Lungs | 3.97E−01 | 1.47E00 | 5.77E−02 | 2.13E−01 |
| Pancreas | 5.09E−01 | 1.88E00 | 7.92E−02 | 2.93E−01 |
| Tumor | 7.85E−01 | 2.90E00 | 3.77E−01 | 1.39E00 |
| Red Marrow | 3.00E−03 | 1.11E−02 | — | — |
| Skeletal Surfaces | 6.07E−01 | 2.25E00 | 5.84E−02 | 2.16E−01 |
| Bone spine | 2.11E−01 | 7.81E−01 | 1.85E−02 | 6.85E−02 |
| Spleen | 3.06E00 | 1.13E01 | 2.30E−01 | 8.51E−01 |
| Thymus | 7.52E−03 | 2.03E−02 | 2.42E−02 | 8.95E−02 |
| Thyroid | 6.10E−03 | 2.26E−02 | 4.10E−03 | 1.52E−02 |
| Urinary Bladder | 8.38E−04 | 3.10E−03 | 4.21E−02 | 1.56E−01 |
| Blood | 2.10E−02 | 7.77E−02 | 2.10E−02 | 7.77E−02 |
| Muscle | 7.90E−02 | 2.92E−01 | 7.90E−02 | 2.92E−01 |
| Total Body | 4.96E−02 | 1.83E−01 | 2.56E−02 | 9.47E−02 |

RIT with 177Lu-IF3 was highly effective in abrogating canine patient derived Gracie tumor growth in SCID mice. FIG. 13 shows the results of the RIT experiment where tumor-bearing mice were treated with 60 μCi 177Lu-IF3 mAb with or without pre-blocking with unlabeled (“cold”) IF3 mAb. There was highly significant abrogation in tumor growth of RIT treated mice in comparison with untreated controls (p=0.0175) and with the “cold” IF3 group (p=0.0025).

The RIT treatment was accompanied by the weight loss in mice treated with RIT which was less pronounced in a group preblocked with “cold” IF3 before RIT administration (FIG. 14). This weight loss necessitated the sacrifice of the mice in RIT group on day 13 after treatment initiation, and in RIT with preblocking group—on Day 18. Analysis of the Kaplan-Meier survival curves (FIG. 15) demonstrated the statistically significant difference (p=0.01) in survival between the RIT and RIT with preblocking groups. The spleens of the mice treated with RIT without pre-blocking with the unlabeled IF3 antibody were somewhat smaller than the spleens of the untreated mice, and some of the mice from RIT group were showing petechiae on their ears (FIG. 16).

Immunohistochemistry of canine spleens demonstrate low expression of IGF2R. Immunohistochemistry of spleens from two dogs performed with IGF2R-specific murine mAb 2G11 demonstrated relatively low expression of IGF2R in comparison with canine placenta used as a positive control (FIG. 17). The staining was observed in the minority of cells, presumably macrophages, and mirrored that of the non-specific control murine mAb MOPC21.

It is noted that the SCID mouse model has an IGF2R-specific limitation of very high expression of this protein by the spleen which may serve as a “sink” of radiolabeled antibody and may have contributed to the toxicity of the treatment. Further, murine FcRn receptors have higher affinity for Fc fragment of human antibodies than for Fc fragment of murine antibodies, thereby leading to shorter half-life [20].

Evaluation of possible toxicity can be assessed for example via nuclear imaging and image based dosimetry in healthy subjects.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely.

The scope of the claims should not be limited by the preferred embodiments and examples, but should be given the broadest interpretation consistent with the description as a whole.

TABLE OF SEQUENCES
SEQ ID NO: 1 nucleotide sequence of VL of 4D5-8
gatatccagatgacccagtccccgagctccctgtccgcctctgtgggcgatagggtcaccatcacctgccgtg
ccagtcagGATGTGAATACCgctgtagcctggtatcaacagaaaccaggaaaagctccgaagcttctgattta
ctcggcatccTTCctctactctggagtcccttctcgcttctctggtagccgttccgggacggatttcactctg
accatcagcagtctgcagccggaagacttcgcaacttattactgtcagcaaCACTACACCACCCCACCAacgt
tcggacagggtaccaaggtggagatcaaa
SEQ ID NO: 2 amino acid sequence of VL of 4D5-8
D I Q M T Q S P S S L S A S V G D R V T I T C R A
S Q D V N T A V A W Y Q Q K P G K A P K L L I Y S
A S F L Y S G V P S R F S G S R S G T D F T L T I
S S L Q P E D F A T Y Y C Q Q H Y T T P P T F G Q
G T K V E I K
SEQ ID NO: 3 nucleotide sequence of VH of 4D5-8
gaggttcagctggtggagtctggcggtggcctggtgcagccagggggctcactccgtttgtcctgtgcagctt
ctggcttcaacattaaggacacctacattcactgggtgcgtcaggccccgggtaagggcctggaatgggttgc
acgcatctacccaaccaatggctacacccgctatgccgatagcgtcaagggccgtttcactataagcgcagac
acatccaaaaacacagcctacctacaaatgaacagcttaagagctgaggacactgccgtctattattgtagcc
gctggggcggcgacggcttttacgccatggactactggggtcaaggaaccctggtcaccgtctcctcg
SEQ ID NO: 4 amino acid sequence of VH of 4D5-8
E V Q L V E S G G G L V Q P G G S L R L S C A A S
G F N I K D T Y I H W V R Q A P G K G L E W V A R
I Y P T N G Y T R Y A D S V K G R F T I S A D T S
K N T A Y L Q M N S L R A E D T A V Y Y C S R W G
G D G F Y A M D Y W G Q G T L V T V S S
SEQ ID NO: 5 4D5-Lib1-L1
CCTGCCGTGCCAGTCAG(N1)HTGTG(N1)HTACCGCTGTAGCCTGGTATC
where (N1) = 10%A, 20%C, 25%G and 45%T
SEQ ID NO: 6 4D5-Lib1-L2
CGAAGCTTCTGATTTAC(N1)HTGCATCC(N1)HTCTCTACTCTGGAGTCC
where (N1) = 10%A, 20%C, 25%G and 45%T
SEQ ID NO: 7 4D5-Lib1-L3-4aa
CTTATTACTGTCAGCAA(N1)HT(N1)HT(N1)HT(N1)HTCCACCAACGTTCGGAC
where (N1) = 10%A, 20%C, 25%G and 45%T
SEQ ID NO: 8 4D5-Lib1-L3-5aa
CTTATTACTGTCAGCAA(N1)HT(N1)HT(N1)HT(N1)HT(N1)HTCCACCAACGTTCGGAC
where (N1) = 10%A, 20%C, 25%G and 45%T
SEQ ID NO: 9 4D5-Lib 1-L3-6aa
CTTATTACTGTCAGCAA(N1)HT(N1)HT(N1)HT(N1)HT(N1)HT(N1)HTCCACCAACGTTCGGAC
where (N1) = 10%A, 20%C, 25%G and 45%T
SEQ ID NO: 10 4D5-Lib1-L3-7aa
CTTATTACTGTCAGCAA(N1)HT(N1)HT(N1)HT(N1)HT(N1)HT(N1)HT(N1)HTCCACCAACGTTCGG
AC
where (N1) = 10%A, 20%C, 25%G and 45%T
SEQ ID NO: 11 4D5-Lib1-H1
GGCTTCAACATTAAG(N1)HTACC(N1)HTATTCACTGGGTGCGTC
where (N1) = 10%A, 20%C, 25%G and 45%T
SEQ ID NO: 12 4D5-Lib1-H2
CTGGAATGGGTTGCA(N1)HTATC(N1)HTCCA(N1)HT(N1)HTGGC(N1)HTACCCGCTATGCCGATAG
where (N1) = 10%A, 20%C, 25%G and 45%T
SEQ ID NO: 13 4D5-Lib1-H3-4aa
CTATTATTGTAGCCGCTGG(N1)HT(N1)HT(N1)HT(N1)HTGCCATGGACTACTGG
where (N1) = 10%A, 20%C, 25%G and 45%T
SEQ ID NO: 14 4D5-Lib1-H3-5aa
CTATTATTGTAGCCGCTGG(N1)HT(N1)HT(N1)HT(N1)HT(N1)HTGCCATGGACTACTGG
where (N1) = 10%A, 20%C, 25%G and 45%T
SEQ ID NO: 15 4D5-Lib1-H3-6aa
CTATTATTGTAGCCGCTGG(N1)HT(N1)HT(N1)HT(N1)HT(N1)HT(N1)HTGCCATGGACTACTGG
where (N1) = 10%A, 20%C, 25%G and 45%T
SEQ ID NO: 16 4D5-Lib1-H3-7aa
CTATTATTGTAGCCGCTGG(N1)HT(N1)HT(N1)HT(N1)HT(N1)HT(N1)HT(N1)HTGCCATGGACTAC
TGG
where (N1) = 10%A, 20%C, 25%G and 45%T
SEQ ID NO: 17 4D5-Lib1-H3-8aa
CTATTATTGTAGCCGCTGG(N1)HT(N1)HT(N1)HT(N1)HT(N1)HT(N1)HT(N1)HT(N1)HTGCCATG
GACTACTGG
where (N1) = 10%A, 20%C, 25%G and 45%T
SEQ ID NO: 18 4D5-Lib1-H3-9aa
CTATTATTGTAGCCGCTGG(N1)HT(N1)HT(N1)HT(N1)HT(N1)HT(N1)HT(N1)HT(N1)HT(N1)HT
GCCATGGACTACTGG
where (N1) = 10%A, 20%C, 25%G and 45%T
SEQ ID NO: 19 4D5-Lib1-H3-10aa
CTATTATTGTAGCCGCTGG(N1)HT(N1)HT(N1)HT(N1)HT(N1)HT(N1)HT(N1)HT(N1)HT(N1)HT
(N1)HTGCCATGGACTACTGG
where (N1) = 10%A, 20%C, 25%G and 45%T
SEQ ID NO: 20 4D5-Lib1-H3-11aa
CTATTATTGTAGCCGCTGG(N1)HT(N1)HT(N1)HT(N1)HT(N1)HT(N1)HT(N1)HT(N1)HT(N1)HT
(N1)HT(N1)HTGCCATGGACTACTGG
where (N1) = 10%A, 20%C, 25%G and 45%T

| SEQ ID | | | Primer |
|---|---|---|---|
| NO: | Primer Name | Primer Sequence* | Direction |
| 21 | MUBV_VK1a | GMCATCCAGATGACCCAGTCTCCATCC | Forward |
| 22 | MUBV_VK1b | GMCATCCAGTTGACCCAGTCTCCATCC | Forward |
| 23 | MUBV_VK1c | GCCATCCGGATGACCCAGTCTCCATYC | Forward |
| 24 | MUBV_Vk1d | GACATCCAGATGACCCAGTCTCCATCTTC | Forward |
| 25 | MUBV_VK1e | GACATCCAGATGACCCAGTCTCCWTCC | Forward |
| 26 | MUBV_VK3a | GAAATTGTGTTGACGCAGTCTCCAGSCACC | Forward |
| 27 | MUBV_VK3b | GAAATAGTGATGACGCAGTCTCCAGCCACC | Forward |
| 28 | MUBV_VK3c | GAAATTGTGTTGACACAGTCTCCAGCC | Forward |
| 29 | MUBV_CHK | ACACTCTCCCCTGTTGAAGCTCTT | Reverse |
| 30 | MUBV_VH3_1L | GAGGTGCAGCTGGTGGAGTCTGGGGGAGGCTTGGTCCAG | Forward |
| 31 | MUBV_VH3_2L | CAGGTGCAGCTGGTGGAGTCTGGGGGAGGCTTGGTCAAG | Forward |
| 32 | MUBV_VH3_3L | GAGGTGCAGCTGGTGGAGTCTGGGGGAGGCTTGGTAAAG | Forward |
| 33 | MUBV_VH3_4L | GAGGTGCAGCTGGTGGAGTCTGGGGGAGGTGTGGTACGG | Forward |
| 34 | MUBV_VH3_5L | CAGGTGCAGCTGGTGGAGTCTGGGGGAGGCGTGGTCCAG | Forward |
| 35 | MUBV_VH3_6L | GAGGTGCAGCTGGTGGAGACTGGAGGAGGCTTGATCCAG | Forward |
| 36 | MUBV_JH1_2 | TGAGGAGACRGTGACCAGGGTG | Reverse |
| 37 | MUBV_JH3 | TGAAGAGACGGTGACCATTGT | Reverse |
| 38 | MUBV_JH4_5 | TGAGGAGACGGTGACCAGGGTT | Reverse |
| 39 | MUBV_JH6 | TGAGGAGACGGTGACCGTGGTCC | Reverse |
| 40 | MUBV_SEC_VK1a | aaatgcctatgcatcg GMC ATC CAG ATG ACC CAG | Forward |
| | | TCT CCA | |
| 41 | MUBV_SEC_VK1b | aaatgcctatgcatcg GMC ATC CAG TTG ACC CAG | Forward |
| | | TCT CCA | |
| 42 | MUBV_SEC_VK1c | aaatgcctatgcatcg GCC ATC CGG ATG ACC CAG | Forward |
| | | TCT CCA | |
| 43 | MUBV_SEC_Vk1d | aaatgcctatgcatcg GAC ATC CAG ATG ACC CAG | Forward |
| | | TCT CCA | |
| 44 | MUBV_SEC_VK1e | aaatgcctatgcatcg GAC ATC CAG ATG ACC CAG | Forward |
| | | TCT CCW | |
| 45 | MUBV_SEC_VK3a | aaatgcctatgcatcg GAA ATT GTG TTG ACG CAG | Forward |
| | | TCT CCA | |
| 46 | MUBV_SEC_VK3b | aaatgcctatgcatcg GAA ATA GTG ATG ACG CAG | Forward |
| | | TCT CCA | |
| 47 | MUBV_SEC_VK3c | aaatgcctatgcatcg GAA ATT GTG TTG ACA CAG | Forward |
| | | TCT CCA | |
| 48 | SAM_SEC_JK1 | GAAGATGAAGACAGATGGTGCAGCCACCGTACGTTTGATT | Reverse |
| | | TCCACCTTGGTCC | |
| 49 | SAM_SEC_JK2 | GAAGATGAAGACAGATGGTGCAGCCACCGTACGTTTGATC | Reverse |
| | | TCCAGCTTGGTCC | |
| 50 | SAM_SEC_JK34 | GAAGATGAAGACAGATGGTGCAGCCACCGTACGTTTGATM | Reverse |
| | | TCCACYTTGGTCC | |
| 51 | SAM_SEC_JK5 | GAAGATGAAGACAGATGGTGCAGCCACCGTACGTTTAATC | Reverse |
| | | TCCAGTCGTGTCC | |
| 52 | SAM_SEC_VH3a | CTTCTTGCATCTATGTTCGTTTTTTCTATTGCTACAAACG | Forward |
| | | CGTATGCTGAGGTGCAGCTGGTGGAG | |
| 53 | SAM_SEC_VH3b | CTTCTTGCATCTATGTTCGTTTTTTCTATTGCTACAAACG | Forward |
| | | CGTATGCTCAGGTGCAGCTGGTGGAG | |
| 54 | MUBV_SEC_JH12 | GGCCTTTTGTGCTAGCTGAGGAGACRGTGACCAG | Reverse |
| 55 | MUBV_SEC_JH3 | GGCCTTTTGTGCTAGCTGAAGAGACGGTGACCAT | Reverse |
| 56 | MUBV_SEC_JH45 | GGCCTTTTGTGCTAGCTGAGGAGACGGTGACCAG | Reverse |
| 57 | MUBV_SEC_JH6 | GGCCTTTTGTGCTAGCTGAGGAGACGGTGACCGT | Reverse |

```
SEQ ID NO: 58 Intergenic sequence encoding constant light (CL) and
internal ribosome entry sites:
cgtacggtggctgcaccatctgtcttcatcttcccgccatctgatgaacagttgaaatctggaactgcctctg
ttgtgtgcctgctgaataacttctatcccagagaggccaaagtacagtggaaggtggataacgccctccaatc
gggtaactcccaggagagtgtcacagagcaggacagcaaggacagcacctacagcctcagcagcaccctgacg
ctgagcaaagcagactacgaaaaacataaagtctacgcctgcgaagtcacccatcagggcctgagctcgcccg
tcacaaagagcttcaacaggggagagtgtggtggttctgattacaaagatgacgatgacaaaTAAttaactcg
aggctgagcaaagcagactactaataacataaagtctacgccggacgcatcgtggccctagtacgcaagttca
cgtaaaaagggtaactagaggttgaggtgattttatgaaaaagaatatcgcatttcttcttgcatctatgttc
gttttttctattgctacaaacgcgtatgct
where bold lowercase represents CL, plain text uppercase indicates a
Stop codon, plain text lowercase indicates noncoding region containing
IRES, and underlined lowercase indicates leader signal peptide of
heavy chain
SEQ ID NO: 59 IGF2R-Fab-1 VL
                                                  CDRL1 CDRL2
AIRMTQSPSSLSASVGDRVTITCRASQDISSWLAWYQQKPDKAPKSLIYAASSLEDGVPSRFSGSGSGTDFTL
TISSLQAEDFATYYCQQYNTYPWTFGQGTKVEIK
                                    CDRL3
SEQ ID NO: 60 IGF2R-Fab-1 VH
                                                            CDRH1 CDRH2
EVQLVESGGGVVQPGGSLRLSCAASGFTFSSYAMHWVRQAPGKGLEWVAVISYDGSNKYYADSVKGRFTISRD
NSKNTLYLQMNSLRAEDTAVYYCARDDRFFGGMDVWGQGTTVTVSS
                                                        CDRH3
SEQ ID NO: 61 IGF2R-Fab-2 VL
                                                  CDRL1 CDRL2
DIQMTQSPSSLSASVGDRVTITCRASQLVDTAVAWYQQKPGKAPKLLIYFASYLYSGVPSRFSGSRSGTDFTL
TISSLQPEDFATYYCQQVVYYPPTFGQGTKVEIK
                                    CDRL3
SEQ ID NO: 62 IGF2R-Fab-2 VH
                                                          CDRH1 CDRH2
EVQLVESGGGLVQPGGSLRLSCAASGFNIKYTYIHWVRQAPGKGLEWVALIDPHYGFTRYADSVKGRFTISAD
TSKNTAYLQMNSLRAEDTAVYYCSRWYYDHAMDYWGQGTLVTVSS
                                                    CDRH3
SEQ ID NO: 63 IGF2R-Fab-3 VL
                                                  CDRL1 CDRL2
DIQMTQSPSSLSASVGDRVTITCRASQDVNTAVAWYQQKPGKAPKLLIYSASFLYSGVPSRFSGSRSGTDFTL
TISSLQPEDFATYYCQQAYFFHHFPPTFGQGTKVEIK
                                      CDRL3
SEQ ID NO: 64 IGF2R-Fab-3 VH
                                                            CDRH1 CDRH2
EVQLVESGGGLVQPGGSLRLSCAASGFNIKDTYIHWVRQAPGKGLEWVAFIDPIFGDTRYADSVKGRFTISAD
TSKNTAYLQMNSLRAEDTAVYYCSRWGGDGFYAMDYWGQGTLVTVSS
                                                      CDRH3
SEQ ID NO: 65 IGF2R-IgG#1-Light Chain
AIRMTQSPSSLSASVGDRVTITCRASQDISSWLAWYQQKPDKAPKSLIYAASSLEDGVPSRFSGSGSGTDFTL
TISSLQAEDFATYYCQQYNTYPWTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
Bold- VL
Underlined- CL (kappa)
SEQ ID NO: 66 IGF2R-IgG#1-Heavy Chain
EVQLVESGGGVVQPGGSLRLSCAASGFTFSSYAMHWVRQAPGKGLEWVAVISYDGSNKYYADSVKGRFTISRD
NSKNTLYLQMNSLRAEDTAVYYCARDDRFFGGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSVMHEALHNHY
TQKSLSLSPGK
Bold- VH
Underlined- IgG1-constant region
SEQ ID NO: 67 IGF2R-IgG#2- Light Chain
DIQMTQSPSSLSASVGDRVTITCRASQLVDTAVAWYQQKPGKAPKLLIYFASYLYSGVPSRFSGSRSGTDFTL
TISSLQPEDFATYYCQQVVYYPPTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
Bold- VL
Underlined- CL (kappa)
SEQ ID NO: 68 IGF2R-IgG#2-Heavy Chain
EVQLVESGGGLVQPGGSLRLSCAASGFNIKYTYIHWVRQAPGKGLEWVALIDPHYGFTRYADSVKGRFTISAD
TSKNTAYLQMNSLRAEDTAVYYCSRWYYDHAMDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYT
QKSLSLSPGK
Bold- VH
Underlined- IgG1-constant region
SEQ ID NO: 69 IGF2R-IgG#3- Light Chain
DIQMTQSPSSLSASVGDRVTITCRASQDVNTAVAWYQQKPGKAPKLLIYSASFLYSGVPSRFSGSRSGTDFTL
TISSLQPEDFATYYCQQAYFFHHFPPTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSFNRGEC
Bold- VL
Underlined- CL (kappa)
SEQ ID NO: 70 IGF2R-IgG#3- Heavy Chain
EVQLVESGGGLVQPGGSLRLSCAASGFNIKDTYIHWVRQAPGKGLEWVAFIDPIFGDTRYADSVKGRFTISAD
TSKNTAYLQMNSLRAEDTAVYYCSRWGGDGFYAMDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPGK
Bold- VH
Underlined- IgG1-constant region
```

| SEQ ID NO: | Sequence Name | Sequence |
|---|---|---|
| SEQ ID NO: 71 | FAB1 CDRL1 | RASQDISSWLA |
| SEQ ID NO: 72 | FAB1 CDRL2 | AASSLED |
| SEQ ID NO: 73 | FAB1 CDRL3 | QQYNTYPWT |
| SEQ ID NO: 74 | FAB1 CDRH1 | SYAMH |
| SEQ ID NO: 75 | FAB1 CDRH2 | VISYDGSNKYYADSVKG |
| SEQ ID NO: 76 | FAB1 CDRH3 | DDRFFGGMDV |
| SEQ ID NO: 77 | FAB2 CDRL1 | RASQLVDTAVA |
| SEQ ID NO: 78 | FAB2 CDRL2 | FASYLYS |
| SEQ ID NO: 79 | FAB2 CDRL3 | QQVVYYPPT |
| SEQ ID NO: 80 | FAB2 CDRH1 | YTYIH |
| SEQ ID NO: 81 | FAB2 CDRH2 | LIDPHYGFTRYADSVKG |
| SEQ ID NO: 82 | FAB2 CDRH3 | SRWYYDHAMDY |
| SEQ ID NO: 83 | FAB3 CDRL1 | RASQDVNTAVA |
| SEQ ID NO: 84 | FAB3 CDRL2 | SASFLYS |
| SEQ ID NO: 85 | FAB3 CDRL3 | QQAYFFHHFPPT |
| SEQ ID NO: 86 | FAB3 CDRH1 | DTYIH |
| SEQ ID NO: 87 | FAB3 CDRH2 | FIDPIFGDTRYADSVKG |
| SEQ ID NO: 88 | FAB3 CDRH3 | SRWGGDGFYAMDY |

SEQ ID NO: 89 Domains 11-13 of human IGF2R
NEHDDCQVTNPSTGHLFDLSSLSGRAGFTAAYSEKGLVYMSICGENENCPPGVGACFGQTRISVGKANKRLRY
VDQVLQLVYKDGSPCPSKSGLSYKSVISFVCRPEAGPTNRPMLISLDKQTCTLFFSWHTPLACEQATECSVRN
GSSIVDLSPLIHRTGGYEAYDESEDDASDTNPDFYINICQPLNPMHGVPCPAGAAVCKVPIDGPPIDIGRVAG
PPILNPIANEIYLNFESSTPCLADKHFNYTSLIAFHCKRGVSMGTPKLLRTSECDFVFEWETPVVCPDEVRMD
GCTLTDEQLLYSFNLSSLSTSTFKVTRDSRTYSVGVCTFAVGPEQGGCKDGGVCLLSGTKGASFGRLQSMKLD
YRHQDEAVVLSYVNGDRCPPETDDGVPCVFPFIFNGKSYEECIIESRAKLWCSTTADYDRDHEWGFCRHSNSY
RTSSIIFKCDEDEDIGRPQVFSEVRGCDVTFEWKTKVVCPP
SEQ ID NO: 90 Domains 11-13 of murine IGF2R
NTHDDCQVTNPSTGHLFDLSSLSGRAGINASYSEKGLVFMSICEENENCGPGVGACFGQTRISVGKASKRLSY
KDQVLQLVYENGSPCPSLSDLRYKSVISFVCRPEAGPTNRPMLISLDKQTCTLFFSWHTPLACEQATECTVRN
GSSIIDLSPLIHRTGGYEAYDESEDDTSDTTPDFYINICQPLNPMHGVPCPAGASVCKVPVDGPPIDIGRVTG
PPIFNPVANEVYLNFESSTHCLADRYMNYTSLITFHCKRGVSMGTPKLIRTNDCDFVFEWETPIVCPDEVKTQ
GCAVTDEQLLYSFNLTSLSTSTFKVTRDARTYSIGVCTAAAGLGQEGCKDGGVCLLSGNKGASFGRLASMQLD
YRHQDEAVILSYVNGDPCPPETDDGEPCVFPFIYKGKSYDECVLEGRAKLWCSKTANYDRDHEWGFCRQTNSY
RMSAIIFTCDESEDIGRPQVFSEDRGCEVTFEWKTKVVCPP
SEQ ID NO: 91 Domains 11-13 of canine IGF2R
NVHDNCQVTNPATGHLFDLSSLSGRAGHTAAYSEKGLVYISICEDNENCSPGVGACFGQTRISVGKANKRLTY
VDQVLQLVYEDGSPCPSKSGLTYKSVISFVCRPEAGPTNRPMLISLDKQTCTLFFSWHTPLACEQVTECSVRN
GSSIIDLSPLIHRTGGYEAYDESEDDTSDTGPDFYINICQPLNPMHGVPCPAGAAVCKVPVDGPPIDIGRVTG
PPILNPIANEVYLNFESSTPCLADKHFNYTSLIAFHCRRGVSMGAPMLLRTSDCDFVFGWETPLVCPDEVKMD
GCSLTDEQLHYSFNLSSLSKSTFKVTRDSRTYSIGVCTAAAGLDEGGCKDGGVCLLSGSKGASFGRLASMRLD
YRHQDEAVILSYANGDNCPPETEAGDPCVFPFIFNGKSYEECVVEGRARLWCSTTANYDRDHEWGFCRHSNSH
RMSSIIFKCDEDADIGRPQVFSEVRGCEVTFEWKTKVVCPP
*Lowercase sequences add restriction site Nsil in frame as a PCR overhang; plain text uppercase sequences indicate annealing regions corresponding to cDNA sequence; bold uppercase sequences indicate the overlap sequence necessary for splicing in SOE PCR reaction. For Seq IDs 54-57, the bold uppercase sequence also includes an Nhel restriction site.

### REFERENCES

Allen K J H, Jiao R, Malo M E, Dadachova E. Evaluation of N-Succinimidyl S-Acetylthioacetate Ligand for Radiolabeling of Humanized Antibodies with ¹⁸⁸Rhenium. Cancer Biother Radiopharm. 2018 Jul 16. doi: 10.1089/cbr.2018.2480. [Epub ahead of print]
Almagro J C, Pedraza-Escalona M, Arrieta H I, Pérez-Tapia S M. Phage Display Libraries for Antibody Therapeutic Discovery and Development. Antibodies (Basel). 2019 Aug 23;8(3):44. doi: 10.3390/antib8030044. PMID: 31544850; PMCID: PMC6784186.
Arslan I, et al The role of insulin-like growth factor I-II receptor on development of pleomorphic adenoma. Eur Arch Otorhinolaryngol. 2017;274(9):3443-3447.
Beatty B G, Kuhn J A, Hui T E, Fisher D R, Williams L E, Beatty J D. Application of the cross-organ beta dose method for tissue dosimetry in tumor-bearing mice treated with a 90Y-labeled immunoconjugate. Cancer 73(3 Suppl):958-65 (1994).
Bharambe H S, et al Downregulation of miR-204 expression defines a highly aggressive subset of Group 3/Group 4 medulloblastomas. Acta Neuropathol Commun. 2019;7(1):52.
Brown J, Delaine C, Zaccheo O J, Siebold C, Gilbert R J, van Boxel G, et al. Structure and functional analysis of the IGF-II/IGF2R interaction. EMBO J. 27(1):265-76 (2008).
Bruland et al. New monoclonal antibodies specific for human sarcomas. Int J Cancer. 1986 Jul 15;38(1):27-31.

Bruland O S, Skretting A, Solheim O P, Aas M. Targeted radiotherapy of osteosarcoma using 153 Sm-EDTMP. A new promising approach. Acta Oncol. 35(3):381-4 (1996).
Bryan R. A, Z. Jiang, T. Jandl, J. Strauss, W. Koba, C. Onyedika, A. Morgenstern, F. Bruchertseifer, R. S. Sellers, A. L. Epstein, E. Dadachova Treatment of experimental pancreatic cancer with 213-Bismuth-labeled chimeric antibody to single-strand DNA. Expert Rev Anticancer Ther. 14(10):1243-9 (2014).
Buss N A, Henderson S J, McFarlane M, Shenton J M, de Haan L. Monoclonal antibody therapeutics: history and future. Curr Opin Pharmacol. 12(5):615-22 (2012).
Camacho X, Calzada V, Fernandez M, Alonso O, Chammas R, Riva E, Gambini J P, Cabral P. 177Lu-DOTA-Bevacizumab: Radioimmunotherapy Agent for Melanoma. Curr Radiopharm. 10(1):21-28 (2017).
Carter, P. et al. Humanization of an anti-p185HER2 antibody for human cancer therapy. Proc Natl Acad Sci U S A 89, 4285-9 (1992).
Cheal S M, Fung E K, Patel M, Xu H, Guo H F, Zanzonico P B, Monette S, Wittrup K D, Cheung N V, Larson S M. Curative Multicycle Radioimmunotherapy Monitored by Quantitative SPECT/CT-Based Theranostics, Using Bispecific Antibody Pretargeting Strategy in Colorectal Cancer J Nucl Med. 58(11):1735-1742 (2017).
Dadachova E., Chappell L. L. and Brechbiel M. W. Spectrophotometric method for determination of bifunctional macrocyclic ligands in macrocyclic ligand—protein conjugates. Nucl. Med. Biol. 26(8), p. 977-982 (1999)
De Martino M C, et al IGF and mTOR pathway expression and in vitro effects of linsitinib and mTOR inhibitors in adrenocortical cancer. Endocrine. 2019;64(3):673-684.
Emami B, Lyman J, Brown A, Coia L, Goitein M, Munzenrider J E, et al. Tolerance of normal tissue to therapeutic irradiation. Int J Radiat Oncol Biol Phys. 21(1):109-22 (1991).
Fellouse F A, Sidhu S S, in Making and using antibodies: A practical handbook (eds. G. C. Howard & M. R. Kaser) 157-180 (CRC Press, Boca Raton, FL; 2007).
Ferrari S, Smeland S, Mercuri M, Bertoni F, Longhi A, Ruggieri P, Alvegard T A, Picci P, Capanna R, Bernini G, Müller C, Tienghi A, Wiebe T, Comandone A, Böhling T, Del Prever A B, Brosjö O, Bacci G, Saeter G; Italian and Scandinavian Sarcoma Groups. Neoadjuvant chemotherapy with high-dose Ifosfamide, high-dose methotrexate, cisplatin, and doxorubicin for patients with localized osteosarcoma of the extremity: A joint study by the Italian and Scandinavian Sarcoma Groups. J Clin Oncol 23: 8845-8852 (2005).
Frenzel A, Kugler J, Wilke S, Schirrmann T, Hust M. Construction of human antibody gene libraries and selection of antibodies by phage display. Methods Mol Biol. 1060:215-43 (2014).
Frenzel A, Schirrmann T, Hust M. Phage display-derived human antibodies in clinical development and therapy. MAbs. 8(7):1177-94 (2016).
Garg R, Allen K J H, Dawicki W, Geoghegan E M, Ludwig D L, Dadachova E. 225Ac-labeled CD33-targeting antibody reverses resistance to Bcl-2 inhibitor venetoclax in acute myeloid leukemia models. Cancer Med. 2020 Dec 21. doi: 10.1002/cam4.3665
Geller DS, J Morris, E Revskaya, M Kahn, W Zhang, S Piperdi, A Park, P Koirala, H Guzik, C Hall, B Hoang, R Yang, M Roth, J Gill, R Gorlick, E Dadachova Targeted therapy of osteosarcoma with radiolabeled monoclonal antibody to an insulin-like growth factor-2 receptor (IGF2R). Nucl. Med. Biol. 43(12):812-817 (2016).
Hughes J, et al Maternal transmission of an Igf2r domain 11: IGF2 binding mutant allele (Igf2r^{I}1565A) results in partial lethality, overgrowth and intestinal adenoma progression. Sci Rep. 2019;9(1):11388.
Lautem A, et al. Expression and prognostic significance of insulin-like growth factor-2 receptor in human hepatocellular carcinoma and the influence of transarterial chemoembolization. Oncol Rep. 2019;41(4):2299-2310.
lida Y, et al. Predominance of triple wild-type and IGF2R mutations in mucosal melanomas. BMC Cancer. 2018;18(1):1054.
Jiang Z., E. Revskaya, D. R. Fisher and E. Dadachova In vivo evaluation of free and chelated accelerator-produced Actinium-225-radiation dosimetry and toxicity results. Curr. Radiopharm. 2018 Apr 23. doi: 10.2174/1874471011666180423120707. [Epub ahead of print]
Gorlick R, Janeway K, Lessnick S, Randall R L, Marina N; COG Bone Tumor Committee. Children's Oncology Group's 2013 blueprint for research: bone tumors. Pediatr Blood Cancer. 60(6):1009-15 (2013).
Hassan S E, Bekarev M, Kim M Y, Lin J, Piperdi S, Gorlick R, Geller D S. Cell surface receptor expression patterns in osteosarcoma. Cancer. 118(3):740-9 (2012).
Haines D M, Bruland O S. Immunohistochemical detection of osteosarcoma-associated antigen in canine osteosarcoma. Anticancer Res. 9(4):903-7 (1989).
Harris L K, I. P. Crocker, P. N. Baker, J. D. Aplin, M. Westwood IGF2 actions on trophoblast in human placenta are regulated by the insulin-like growth factor 2 receptor, which can function as both a signaling and clearance receptor. Biol Reprod. 84(3):440-6 (2011).
Houghton P J, Morton C L, Tucker C, Payne D, Favours E, Cole C, Gorlick R, Kolb E A, Zhang W, Lock R, Carol H, Tajbakhsh M, Reynolds C P, Maris J M, Courtright J, Keir S T, Friedman H S, Stopford C, Zeidner J, Wu J, Liu T, Billups C A, Khan J, Ansher S, Zhang J, Smith M A. The pediatric preclinical testing program: description of models and early testing results. Pediatr Blood Cancer. 49(7):928-40 (2007).
Hust, M., Frenzel, A., Meyer, T., Schirrmann, T. & Dubel, S. Construction of human naive antibody gene libraries. Methods Mol Biol 907, 85-107 (2012).
Karkare S, Allen K J H, Jiao R, Malo M E, Dawicki W, Helal M, Godson D L, Dickinson R, MacDonald-Dickinson V, Yang R, Hoang B, Gorlick R, Geller D S, Dadachova E. Detection and targeting insulin growth factor receptor type 2 (IGF2R) in osteosarcoma PDX in mouse models and in canine osteosarcoma tumors. Sci Rep. 9(1):11476 (2019).
Kim S, Park I, Park S G, Cho S, Kim J H, Ipper N S, et al. Generation, Diversity Determination, and Application to Antibody Selection of a Human Naive Fab Library. Mol Cells. 40(9):655-66 (2017).
Larson, S. M., Carrasquillo, J. A., Cheung, N. V., Press, O. W. Radioimmunotherapy of human tumours. Nature 15, 347-360 (2015).
Kaminski M S, Tuck M, Estes J, Kolstad A, Ross C W, Zasadny K, et al. 131I-tositumomab therapy as initial treatment for follicular lymphoma. N Engl J Med 352:441-9 (2005).
Ladanyi M, Gorlick R. Molecular pathology and molecular pharmacology of osteosarcoma. Pediatr Pathol Lab Med 19: 391-413 (2000).
Larsen R H, Bruland O S, Hoff P, Alstad J, Lindmo T, Rofstad E K. Inactivation of human osteosarcoma cells in vitro by 211At-TP-3 monoclonal antibody: comparison with astatine-211-labeled bovine serum albumin, free astatine-211 and external-beam X rays. Radiat Res. 139(2):178-84 (1994).
Lee E C, Liang Q, Ali H, Bayliss L, Beasley A, Bloomfield-Gerdes T, et al. Complete humanization of the mouse immunoglobulin loci enables efficient therapeutic antibody discovery. Nat Biotechnol. 32(4):356-63 (2014).
Meyers P A, Schwartz C L, Krailo M, Kleinerman E S, Betcher D, Bernstein M L, Conrad E, Ferguson W, Gebhardt M, Goorin A M, Harris M B, Healey J, Huvos A, Link M, Montebello J, Nadel H, Nieder M, Sato
J, Siegal G, Weiner M, Wells R, Wold L, Womer R, Grier H. Osteosarcoma: A randomized, prospective trial of the addition of ifosfamide and/or muramyl tripeptide to cisplatin, doxorubicin, and high-dose methotrexate. J Clin Oncol 23: 2004-2011 (2005).
Milenic D E, Brady E D, Brechbiel M W. Antibody-targeted radiation cancer therapy. Nature Rev Drug Discovery 3: 488-99 (2004).
Molina-Trinidad E M, de Murphy C A, Ferro-Flores G, Murphy-Stack E, Jung-Cook H, “Radiopharmacokinetic and dosimetric parameters of Re-188-lanreotide in athymic mice with induced human cancer tumors,” Int. J. Pharm. 310:125-30 (2006).
Nelson A L, Dhimolea E, Reichert J M. Development trends for human monoclonal antibody therapeutics. Nat Rev Drug Discov. 9(10):767-74 (2010).
Olimpieri P P, Marcatili P, Tramontano A. Tabhu: tools for antibody humanization. Bioinformatics. 31(3):434-5 (2015).
Persson, H. et al. CDR-H3 diversity is not required for antigen recognition by synthetic antibodies. J Mol Biol 425, 803-11 (2013).
Phaeton R, J. Gutierrez, Z. Jiang, R. G. Karabakhtsian, J. Albanese, J. Sunkara, D. R. Fisher, G. L. Goldberg, and E. Dadachova Naïve and radiolabeled antibodies to E6 and E7 HPV-16 oncoproteins show pronounced antitumor activity in experimental cervical cancer. Immunother. 7(6):631-40 (2015).
Phaeton R, Jiang Z, Revskaya E, Fisher D R, Goldberg G L, and Dadachova E. Beta emitters Rhenium-188 and Lutetium-177 are equally effective in radioimmunotherapy of HPV-positive experimental cervical cancer. Cancer Med. 5(1):9-16 (2016).
Popwell S J, Schulz M D, Wagener K B, Batich C D, Milner R J, Lagmay J, Bolch W E. Synthesis of polymeric phosphonates for selective delivery of radionuclides to osteosarcoma. Cancer Biother Radiopharm. 29(7):273-82 (2014).
Prydz K, Brändli A W, Bomsel M, Simons K. Surface distribution of the mannose 6-phosphate receptors in epithelial Madin-Darby canine kidney cells. J Biol Chem.25;265(21):12629-35 (1990).
Reddy S T, Chai W, Childs R A, Page J D, Feizi T, Dahms N M. Identification of a low affinity mannose 6-phosphate-binding site in domain 5 of the cation-independent mannose 6-phosphate receptor. J Biol Chem. 279(37):38658-67 (2004).
Repetto-Llamazares A H, Larsen R H, Giusti A M, Riccardi E, Bruland ØS, Selbo P K, Dahle J 177Lu-DOTA-HH1, a novel anti-CD37 radio-immunoconjugate: a study of toxicity in nude mice. PLoS One. 9(7):e103070 (2014).
Revskaya E., Z. Jiang, A. Morgenstern, F. Bruchertseifer, M. Sesay, S. Walker, S. Fuller, M. S. Lebowitz, C. Gravekamp, H. A. Ghanbari, and E. Dadachova A radiolabeled fully human antibody to human aspartyl (asparaginyl) beta-hydroxylase (HAAH) is a promising agent for imaging and therapy of metastatic breast cancer. Cancer Biother. Radiopharm., 32(2):57-65 (2017).
Riccardo F, Aurisicchio L, Impellizeri J A, Cavallo F. The importance of comparative oncology in translational medicine. Cancer Immunol Immunother. 64(2):137-48 (2015).
Riedl T, van Boxtel E, Bosch M, Parren P W, Gerritsen A F. High-Throughput Screening for Internalizing Antibodies by Homogeneous Fluorescence Imaging of a pH-Activated Probe. J Biomol Screen. 21(1):12-23 (2016).
Ries L A, Smith M A S, Gurney J G, et al (Eds), SEER program, National Cancer Institute, Bethesda, MD (1999).
Savage S A, Woodson K, Walk E, Modi W, Liao J, Douglass C, Hoover R N, Chanock S J; National Osteosarcoma Etiology Study Group. Analysis of genes critical for growth regulation identifies Insulin-like Growth Factor 2 Receptor variations with possible functional significance as risk factors for osteosarcoma. Cancer Epidemiol Biomarkers Prey. 16(8):1667-74 (2007).
Schiffman J D, Breen M. Comparative oncology: what dogs and other species can teach us about humans with cancer. Philos Trans R Soc Lond B Biol Sci. 19;370(1673) (2015).
Schwimmer L J, Huang B, Giang H, Cotter R L, Chemla-Vogel D S, Dy F V, et al. Discovery of diverse and functional antibodies from large human repertoire antibody libraries. J Immunol Methods. 391(1-2):60-71 (2013).
Severi S, Grassi I, Nicolini S, Sansovini M, Bongiovanni A, Paganelli G. Peptide receptor radionuclide therapy in the management of gastrointestinal neuroendocrine tumors: efficacy profile, safety, and quality of life. Onco Targets Ther. 27;10:551-557 (2017).
Smith M A, Gurney J G, Ries L A. Cancer in adolescents 15 to 19 years old. In: Cancer Incidence and Survival Among Children and Adolescents: United States SEER Program 1975-1995 (Pub #99-4649)
Stabin M G, Sparks R B, and Crowe E, “OLINDA/EXM: the second-generation personal computer software for internal dose assessment in nuclear medicine, J Nucl Med 46: 1023-7 (2005).
Vazquez-Lombardi, R. et al. Transient expression of human antibodies in mammalian cells. Nat Protoc 13, 99-117 (2018).
Veterinary cooperative oncology group—common terminology criteria for adverse events (VCOG-CTCAE) following chemotherapy or biological antineoplastic therapy in dogs and cats v1.1. Vet Comp Oncol. (2011)
Wang J, Danska J, Dick J, Sidhu S S, Uppalapati M. Antibodies and antibody fragments targeting SIRP-alpha and their use in treating hematologic cancers, 2014 Patent US 2014/0242095 A1
Wedekind M F, Wagner L M, Cripe T P Immunotherapy for osteosarcoma: Where do we go from here? Pediatr Blood Cancer. 65(9):e27227 (2018).
WestrØm S, BØnsdorff T B, Abbas N, Bruland ØS, Jonasdottir T J, Mælandsmo G M, Larsen R H. Evaluation of CD146 as Target for Radioimmunotherapy against Osteosarcoma. PLoS One. 11(10):e0165382 (2016).

1. An antibody which specifically binds an epitope in domains 11-13 of human, murine, and canine insulin-like growth factor-2 receptor (IGF2R), domains 11-13 of human, murine, and canine IGF2R having the amino acid sequence as set forth in SEQ ID NOs: 89, 90, and 91, wherein the antibody binds human, murine, and/or canine IGF2R with at least or about 2-fold, at least or about 3-fold, at least or about 4-fold, or at least or about 5-fold greater affinity than monoclonal mouse antibody 2G11 as determined by flow cytometry using OS cells selected from OS33 cells, McKinley cells, and/or Gracie cells.

2. The antibody of claim 1, comprising a light chain variable region and a heavy chain variable region, the light chain variable region comprising complementarity determining regions CDR-L1, CDR-L2, and CDR-L3, and the heavy chain variable region comprising complementarity determining regions CDR-H1, CDR-H2, and CDR-H3, wherein the amino acid sequences of said CDRs are:

| | CDR-L1 |
|---|---|
| (SEQ ID NO: 71) | |
| | RASQDISSWLA; |
| | CDR-L2 |
| (SEQ ID NO: 72) | |
| | AASSLED; |
| | CDR-L3 |
| (SEQ ID NO: 73) | |
| | QQYNTYPWT; |
| | CDR-H1 |
| (SEQ ID NO: 74) | |
| | SYAMH; |
| | CDR-H2 |
| (SEQ ID NO: 75) | |
| | VISYDGSNKYYADSVKG; |
| | and |
| | CDR-H3 |
| (SEQ ID NO: 76) | |
| | DDRFFGGMDV; |
| | CDR-L1 |
| (SEQ ID NO: 77) | |
| | RASQLVDTAVA; |
| | CDR-L2 |
| (SEQ ID NO: 78) | |
| | FASYLYS; |
| | CDR-L3 |
| (SEQ ID NO: 79) | |
| | QQVVYYPPT; |
| | CDR-H1 |
| (SEQ ID NO: 80) | |
| | YTYIH; |
| | CDR-H2 |
| (SEQ ID NO: 81) | |
| | LIDPHYGFTRYADSVKG; |
| | and |
| | CDR-H3 |
| (SEQ ID NO: 82) | |
| | SRWYYDHAMDY; |
| | or |
| | CDR-L1 |
| (SEQ ID NO: 83) | |
| | RASQDVNTAVA; |
| | CDR-L2 |
| (SEQ ID NO: 84) | |
| | SASFLYS; |
| | CDR-L3 |
| (SEQ ID NO: 85) | |
| | QQAYFFHHFPPT; |
| | CDR-H1 |
| (SEQ ID NO: 86) | |
| | DTYIH; |
| | CDR-H2 |
| (SEQ ID NO: 87) | |
| | FIDPIFGDTRYADSVKG; |
| | and |
| | CDR-H3 |
| (SEQ ID NO: 88) | |
| | SRWGGDGFYAMDY. |

3. The antibody of claim 2, wherein the light chain variable region and heavy chain variable region comprise i) a polypeptide having an amino acid sequence of SEQ ID NOs: 59 and 60; SEQ ID NOs: 61 and 62; or SEQ ID NOs: 63 and 64; ii) a polypeptide having an amino acid sequence with at least 80%, at least 90%, or at least 95% sequence identity to SEQ ID NOs: 59 and 60; SEQ ID NOs: 61 and 62; or SEQ ID NOs: 63 and 64 wherein the CDR sequences are those shown underlined therein; or iii) a conservatively substituted amino acid sequence of i) wherein the CDR sequences are those shown underlined therein.

4. The antibody of any one of claims 1 to 3, wherein the antibody is a humanized or human antibody.

5. The antibody of any one of claims 1 to 4, wherein the antibody is a single chain antibody.

6. The antibody of any one of claims 1 to 5, wherein the antibody is an antibody fragment selected from Fab, Fab′, F(ab′)2, scFv, dsFv, ds-scFv, dimers, nanobodies, minibodies, diabodies, and multimers thereof.

7. The antibody of any one of claims 1 to 5, wherein the antibody is an IgG, optionally IgG1.

8. The antibody of claim 7, wherein the antibody comprises a light chain and a heavy chain the light chain and heavy chain comprising i) a polypeptide having an amino acid sequence of SEQ ID NO: 65 and 66, SEQ ID NO: 67 and 68, or SEQ ID NO: 69 and 70 respectively; ii) a polypeptide having an amino acid sequence with at least 80%, at least 90%, or at least 95% sequence identity to SEQ ID NO: 65 and 66, SEQ ID NO: 67 and 68, or SEQ ID NO: 69 and 70 wherein the CDR sequences are those of SEQ ID NOs: 71-75, SEQ ID NOs: 77-82, or SEQ ID NOs: 83-88; or iii) a conservatively substituted amino acid sequence of i) wherein the CDR sequences are those of SEQ ID NOs: 71-75, SEQ ID NOs: 77-82, or SEQ ID NOs: 83-88.

9. The antibody of claim 8, wherein the light chain comprises a polypeptide having an amino acid sequence of SEQ ID NO: 69 and the heavy chain comprise a polypeptide having an amino acid sequence of SEQ ID NO: 70.

10. The antibody of any one of claims 1 to 9, wherein the antibody competes for binding to domains 11-13 of human, murine, or canine insulin-like growth factor-2 receptor (IGF2R) with an antibody comprising the CDR sequences of claim 2, the light chain variable and heavy chain variable sequences of claim 4, or the light chain and heavy chain sequences of claim 9 or 10.

11. A nucleic acid molecule encoding the antibody of any one of claims 1 to 10.

12. A vector comprising the nucleic acid molecule of claim 11.

13. A cell comprising the nucleic acid molecule of claim 11, the vector of claim 12, or expressing the antibody of any one of claims 1 to 10.

14. An immunoconjugate comprising the antibody of any one of claims 1 to 10 and a therapeutic agent, and/or detectable label.

15. The immunoconjugate of claim 14, wherein the detectable label and/or therapeutic agent is a radionuclide, optionally an alpha- beta- or gamma-emitting radionuclide.

16. The immunoconjugate of claim 15, wherein the antibody is labeled with the radionuclide using a bifunctional linker, optionally CHXA″, optionally at a chelator-antibody ratio (CAR) of about 0.7 to about 1.3, about 0.8 to about 1.2, about 0.9, or about 1.

17. The immunoconjugate of claim 15 or claim 16 wherein the radionuclide is selected from 223Radium, 177Lutetium, 188Rhenium, 111Indium, and 225Actinium.

18. The immunoconjugate of claim 17, wherein the antibody is the antibody of claim 9 and the radionuclide is 111Indium or 177Lutetium.

19. A composition comprising the antibody of any one of claims 1-10, the nucleic acid molecule of claim 11, the vector of claim 12, the cell of claim 13, or the immunoconjugate of any one of claims 14 to 18, and a diluent or pharmaceutically acceptable carrier.

20. The antibody of any one of claims 1-9, the immunoconjugate of any one of claims 14 to 18, or the composition of claim 19, for use in treating cancer, optionally the cancer is selected from osteosarcoma, pleomorphic adenoma, intestinal adenoma, medulloblastoma, adenocortical carcinoma, mucosal melanoma, and hepatocellular carcinoma, preferably the cancer is osteosarcoma, in a subject in need thereof.

21. The antibody, immunoconjugate, or composition for use of claim 20, wherein the subject is a human or a canine.

22. A method for detecting IGF2R expression in a biological sample, the method comprising a) obtaining a biological sample suspected of containing IGF2R, b) contacting the sample with the antibody of any one of claims 1 to 10 or the immunoconjugate of any one of claims 14 to 18 under conditions permissive for forming an antibody:IGF2R complex, and c) detecting the presence of any complex, wherein the presence of detectable complex is indicative that the sample expresses IGF2R.

23. A method of detecting whether a subject has an IGF2R-expressing cancer, the method comprising obtaining a biological sample suspected of containing an IGF2R-expressing cancer cell from the subject, contacting the sample with the antibody of any one of claims 1 to 10 or the immunoconjugate of any one of claims 14 to 18 under conditions permissive for forming an antibody:IGF2R complex, and detecting the presence of an antibody complex, wherein the presence of an antibody complex indicates that the subject has an IGF2R-expressing cancer, optionally the IGF2R-expressing cancer is selected from osteosarcoma, pleomorphic adenoma, intestinal adenoma, medulloblastoma, adenocortical carcinoma, mucosal melanoma, and hepatocellular carcinoma, optionally the IGF2R-expressing cancer is osteosarcoma.

24. The method of claim 23, wherein the biological sample is obtained from a subject having or suspected of having a cancer, optionally osteosarcoma, optionally the biological sample is a tumor sample.

25. A method for imaging an IGF2R-expressing tumor in a subject, the method comprising administering the antibody of any one of claims 1 to 10, the immunoconjugate of any one of claims 14 to 18, or the composition of claim 19 to the subject, and detecting the presence of the label, optionally the antibody is an IgG.

26. A method of determining if a subject has an IGF2R-expressing tumor, the method comprising administering the antibody of any one of claims 1 to 10, the immunoconjugate of any one of claims 14 to 18, or the composition of claim 19 to the subject, and detecting the presence of the label by imaging, optionally the subject has or is suspected of having an IGF2R-expressing cancer, optionally the IGF2R-expressing cancer is selected from osteosarcoma, pleomorphic adenoma, intestinal adenoma, medulloblastoma, adenocortical carcinoma, mucosal melanoma, and hepatocellular carcinoma, preferably the IGF2R-expressing cancer is osteosarcoma.

27. The method of claim 25 or 26, wherein the subject is a human or a canine.

28. The method of any one of claims 25 to 27, wherein the immunoconjugate is the immunoconjugate of claim 18 or the composition comprises the immunoconjugate of claim 18.

29. A method of treating a cancer, optionally the cancer is selected from osteosarcoma, pleomorphic adenoma, intestinal adenoma, medulloblastoma, adenocortical carcinoma, mucosal melanoma, and hepatocellular carcinoma, preferably the cancer is osteosarcoma, in a subject in need thereof, the method comprising administering an effective amount of the antibody of any one of claims 1 to 10, the immunoconjugate of any one of claims 14 to 18, or the composition of claim 19 to the subject, preferably the antibody is an IgG.

30. The method of claim 29, wherein the subject is a human or a canine.

31. The method of claim 29 or claim 30, wherein the immunoconjugate is the immunoconjugate of claim 18 or the composition comprises the immunoconjugate of claim 18.

32. The method of any one of claims 29 to 31, wherein the method further comprises a) detecting IGF2R expression in a biological sample according to the method of any one of claims 22 to 24, wherein the biological sample is obtained from the subject, and/or b) imaging an IGF2R-expressing tumor in the subject according to the method of claim 25 or claim 26, wherein the detecting and/or imaging is done before, during, or following administering the antibody, immunoconjugate, or composition.

33. A library comprising nucleic acid molecules encoding antibodies based on a 4D5-8 framework, the antibodies comprising one or more mutations to solvent exposed CDR residues identified in FIG. 1.
